SEQUENCE LISTING (50 sequences)

SEQ ID NO: 1 - protein, 20 aa, Plasmodium species
MPIPNNPGAGENAFDPVFVK

SEQ ID NO: 2 - protein, 20 aa, Plasmodium species
ENAFDPVFVKDDDGYDLDSF

SEQ ID NO: 3 - protein, 20 aa, Plasmodium species
DDDGYDLDSFMIPAHYKKYL

SEQ ID NO: 4 - protein, 20 aa, Plasmodium species
MIPAHYKKYLTKVLVPNGVI

SEQ ID NO: 5 - protein, 20 aa, Plasmodium species
TKVLVPNGVIKNRIEKLAYD

SEQ ID NO: 6 - protein, 20 aa, Plasmodium species
KNRIEKLAYDIKKVYNNEEF

SEQ ID NO: 7 - protein, 20 aa, Plasmodium species
IKKVYNNEEFHILCLLKGSR

SEQ ID NO: 8 - protein, 20 aa, Plasmodium species
HILCLLKGSRGFFTALLKHL

SEQ ID NO: 9 - protein, 20 aa, Plasmodium species
GFFTALLKHLSRIHNYSAVE

SEQ ID NO: 10 - protein, 20 aa, Plasmodium species
SRIHNYSAVEMSKPLFGEHY

SEQ ID NO: 11 - protein, 20 aa, Plasmodium species
MSKPLFGEHYVRVKSYCNDQ

SEQ ID NO: 12 - protein, 20 aa, Plasmodium species
VRVKSYCNDQSTGTLEIVSE

SEQ ID NO: 13 - protein, 20 aa, Plasmodium species
STGTLEIVSEDLSCLKGKHV

SEQ ID NO: 14 - protein, 20 aa, Plasmodium species
DLSCLKGKHVLIVEDIIDTG

SEQ ID NO: 15 - protein, 20 aa, Plasmodium species
LIVEDIIDTGKTLVKFCEYL

SEQ ID NO: 16 - protein, 20 aa, Plasmodium species
KTLVKFCEYLKKFEIKTVAI

SEQ ID NO: 17 - protein, 20 aa, Plasmodium species
KKFEIKTVAIACLFIKRTPL

SEQ ID NO: 18 - protein, 20 aa, Plasmodium species
ACLFIKRTPLWNGFKADFVG

SEQ ID NO: 19 - protein, 20 aa, Plasmodium species
WNGFKADFVGFSIPDHFVVG

SEQ ID NO: 20 - protein, 20 aa, Plasmodum species
FSIPDHFVVGYSLDYNEIFR

SEQ ID NO: 21 - protein, 20 aa, Plasmodium species
YSLDYNEIFRDLDHCCLVND

SEQ ID NO: 22 - protein, 21 aa, Plasmodium species
DLDHCCLVNDEGKKKYKATSL

SEQ ID NO: 23 - protein, 20 aa, Plasmodium species
MPIPNNPGAGENAFDPVFVN

SEQ ID NO: 24 - protein, 20 aa, Plasmodium species
TSKPLFGEHYVRVKSYCNDQ

SEQ ID NO: 25 - DNA, 60 bp, Plasmodium sp.
atgccaataccaaataatccaggagctggtgaaaatgcctttgatcccgttttcgtaaag

SEQ ID NO: 26 - DNA, 60 bp, Plasmodium sp.
gaaaatgcctttgatcccgttttcgtaaaggatgacgatggttatgaccttgattctttt

SEQ ID NO: 27 - DNA, 60 bp, Plasmodium sp.
gatgacgatggttatgaccttgattcttttatgatccctgcacattataaaaaatatctt

SEQ ID NO: 28 - DNA, 60 bp, Plasmodium sp.
atgatccctgcacattataaaaaatatcttaccaaggtcttagttccaaatggtgtcata

SEQ ID NO: 29 - DNA, 60 bp, Plasmodium sp.
accaaggtcttagttccaaatggtgtcataaaaaaccgtattgagaaattggcttatgat

SEQ ID NO: 30 - DNA, 60 bp, Plasmodium sp.
aaaaaccgtattgagaaattggcttatgatattaaaaaggtgtacaacaatgaagagttt

SEQ ID NO: 31 - DNA, 60 bp, Plasmodium sp.
attaaaaaggtgtacaacaatgaagagtttcatattctttgtttgttgaaaggttctcgt

SEQ ID NO: 32 - DNA, 60 bp, Plasmodium sp.
catattctttgtttgttgaaaggttctcgtggttttttcactgctctcttaaagcattta

SEQ ID NO: 33 - DNA, 60 bp, Plasmodium sp.
ggttttttcactgctctcttaaagcatttaagtagaatacataattatagtgccgttgag

SEQ ID NO: 34 - DNA, 60 bp, Plasmodium sp.
agtagaatacataattatagtgccgttgagatgtccaaaccattatttggagaacactac

SEQ ID NO: 35 - DNA, 60 bp, Plasmodium sp.
atgtccaaaccattatttggagaacactacgtacgtgtgaaatcctattgtaatgaccaa

SEQ ID NO: 36 - DNA, 60 bp, Plasmodium sp.
gtacgtgtgaaatcctattgtaatgaccaatcaacaggtacattagaaattgtaagtgaa

SEQ ID NO: 37 - DNA, 60 bp, Plasmodium sp.
tcaacaggtacattagaaattgtaagtgaagatttatcttgtttaaaaggaaaacatgta

SEQ ID NO: 38 - DNA, 60 bp, Plasmodium sp.
gatttatcttgtttaaaaggaaaacatgtattaattgttgaagatattattgatactggt

SEQ ID NO: 39 - DNA, 60 bp, Plasmodium sp.
ttaattgttgaagatattattgatactggtaaaacattagtaaagttttgtgaatactta

SEQ ID NO: 40 - DNA, 60 bp, Plasmodium sp.
aaaacattagtaaagttttgtgaatacttaaagaaatttgaaataaaaaccgttgccatc

SEQ ID NO: 41 - DNA, 60 bp, Plasmodium sp.
aagaaatttgaaataaaaaccgttgccatcgcttgtctttttattaaaagaacacctttg

SEQ ID NO: 42 - DNA, 60 bp, Plasmodium sp.
gcttgtctttttattaaaagaacacctttgtggaatggttttaaagctgatttcgttgga

SEQ ID NO: 43 - DNA, 60 bp, Plasmodium sp.
tggaatggttttaaagctgatttcgttggattctcaattcctgatcactttgttgttggt

SEQ ID NO: 44 - DNA, 60 bp, Plasmodium sp.
ttctcaattcctgatcactttgttgttggttatagtttagactataatgaaattttcaga

SEQ ID NO: 45 - DNA, 60 bp, Plasmodium sp.
tatagtttagactataatgaaattttcagagatcttgaccattgttgtttggttaatgat

SEQ ID NO: 46 - DNA, 63 bp, Plasmodium sp.
gatcttgaccattgttgtttggttaatgatgagggaaaaaagaaatataaagcaacttcatta

SEQ ID NO: 47 - DNA, 60 bp, Plasmodium sp.
atgccaataccaaataatccaggagctggtgaaaatgcctttgatcccgttttcgtaaac

SEQ ID NO: 48 - DNA, 60 bp, Plasmodium sp.
acgtccaaaccattatttggagaacactacgtacgtgtgaaatcctattgtaatgaccaa

SEQ ID NO: 49 - protein, 19 aa, Plasmodium yoelii
MKIPNNPGAGELGYEPVMI

SEQ ID NO: 50 - protein, 5 aa, Plasmodium yoelii
MKIPN

(19) United States
(12) Patent Application Publication
(10) Pub. No.: US 2005/0123557 A1
(43) Pub. Date: Jun. 9, 2005

(54) AntiI-protozoal vaccine

(75) Inventors: Morris Makobongo, Herston (AU); George Alfred Riding, Yeronga (AU); Peter Willadsen, Chapel Hill (AU); Michael Good, The Gap (AU)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: The Council of hte Queensland Institute of Medical Research, Herston (AU)

(21) Appl. No.: 10/506,053

(22) PCT Filed: Feb. 28, 2003
(86) PCT No.: PCT/AU03/00246

(30) Foreign Application Priority Data
Mar. 1, 2002 (AU) PS 0845

(51) Int. Cl. C07H 21/04; A61K 39/002; C12N 15/74; C12N 9/10

(52) U.S. Cl. 424/191.1; 530/388.6; 435/193; 435/69.3; 435/320.1; 435/258.2; 536/23.2

(57) ABSTRACT

Immunotherapy of protozoal diseases is provided by use of hypoxanthine guanine xanthine phosphoribosyl transferase protein, or peptide fragments thereof, as an immunogen in vaccines effective against protozoal diseases such as malaria and babesiosis. In particular, immunization with hypoxanthine guanine xanthine phosphoribosyl transferase or peptide fragments thereof, induces T cell immunity to blood stage malaria. In particular embodiments, the invention provides protein and DNA malaria vaccines and methods of prophylactic and therapeutic immunization that elicit T cell-mediated immune responses broadly applicable to protozoal diseases including malaria.

### FIELD OF THE INVENTION

THIS INVENTION relates to immunotherapy of protozoal diseases. More particularly, this invention relates to use of hypoxanthine guanine xanthine phosphoribosyl transferase protein, or peptide fragments thereof, as an immunogen in vaccines effective against protozoal diseases such as malaria and babesiosis. A surprising feature of this invention is that immunization with hypoxanthine guanine xanthine phosphoribosyl transferase or peptide fragments thereof, induces T cell immunity to blood stage malaria. The invention therefore provides protein and DNA malaria vaccines and methods of prophylactic and therapeutic immunization that elicit T cell-mediated immune responses broadly applicable to protozoal diseases including malaria.

### BACKGROUND OF THE INVENTION

Protozoal diseases in general have enormous impact on the health and survival of humans and animals, in particular domestic livestock and companion animals. Disease organisms include those causing malaria in humans (Plasmodium spp.), Babesis bovis, Babesia bigemina and Babesia divergens infecting cattle, Babesia canis infecting dogs, Babesia microti and Babesia divergens infecting humans. Major diseases caused by other protozoal species include, as examples, Trypanosomiasis in man and animals, especially cattle (Trypanosoma gambiense, T. congolense, T. cruzi and other species), the various forms of Leishmaniasis (Leishmania donovani, L. tropica, L. brasiliensis, L. mexicana) Giardiasis (Giardia lamblia), Toxoplasmosis (Toxoplasma gondii). In addition, in animal production coccidiosis due to Eimeria tenella in chickens has a severe economic impact.

Malaria is a major cause of morbidity and mortality in humans, especially in underdeveloped countries. Malaria affects 400-500 million people world wide and is responsible for over two million deaths per year (Murray et al., 1995, Manual of Clinical Microbiology (6th ed.) ASM Press, Washington D.C.).

Malaria is caused by protozoan parasites of the genus Plasmodium. Four species affect humans, P. falciparum, P. vivax, P. malariae and P. ovale. The malaria parasite has a complex life cycle which includes a blood stage where asexual eproduction occurs within erythrocytes of a vertebrate host.

Free parasites (merozoites) recognise, attach to and invade the erythrocytes. Once internalised, the parasite replicates, forms schizonts and newly developed merozoites are released from the infected erythrocyte into the plasma where they may infect other erythrocytes.

Existing prevention or treatment methods, including drugs for application to humans to kill parasites and insecticides to kill mosquito vectors, are increasingly problematic due to increased drug and insecticide resistance.

A malaria vaccine will be an important addition to existing malaria control programs in endemic regions and for travellers with short term exposure to malaria.

Attempts to develop a malaria vaccine have focussed on identifying suitable malaria parasite proteins to initiate an appropriate host immune response. The complex life cycle of the malaria parasite, however, has made identification of such proteins a difficult task.

Although there is good evidence that immunity to the blood stages of malaria parasites can be mediated by different effector components of the adaptive immune system (Freeman & Parish, 1981; Grun & Weidanz, 1983; Brake et al, 1988; Seixas & Langhorne, 1999), target antigens for a principal component, effector CD4⁺ T cells, have never been defined. To date only target antigens for antibodies have been defined and these targets are either variant, with the ability to vary within a clone, or demonstrate allelic polymorphism (Good, 2001).

### SUMMARY OF THE INVENTION

The present invention is broadly directed to use of hypoxanthine guanine xanthine phosphoribosyl transferase (HGXPRT) protein and/or immunogenic fragments thereof, as an immunogen that is capable of eliciting an immune response to a protozoan organism.

Preferably, the HGXPRT protein, immunogenic fragment thereof or encoding nucleic acid is isolated from a Plasmodium species or a Babesia species.

In a first aspect, the invention provides an isolated protein comprising at least one immunogenic fragment of hypoxanthine guanine xanthine phosphoribosyl transferase (HGXPRT) protein, wherein the isolated protein is not a full-length HGXPRT.

In a particular embodiment, the isolated protein comprises an amino acid sequence selected from the group consisting of: SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO:10; SEQ ID NO:11; SEQ ID NO:12; SEQ ID NO:13; SEQ ID NO:14; SEQ ID NO:15; SEQ ID NO:16; SEQ ID NO:17; SEQ ID NO:18; SEQ ID NO:19; SEQ ID NO:20; SEQ ID NO:21; SEQ ID NO:22; SEQ ID NO:23; and SEQ ID NO:24.

Preferred amino acid sequences are set forth in SEQ ID NOS: 6, 7, 8, 9, 10, 11, 12, 15, 17, 18, 21, 22, 23 and 24.

More preferred amino acid sequences are set forth in SEQ ID NOS: 17 and 21.

In another particular embodiment, the invention provides an isolated protein comprising a plurality of immunogenic fragments of a HGXPRT protein.

This aspect of the invention also contemplates variants and derivatives of the aforementioned HGXPRT fragments and isolated proteins.

In a second aspect, the invention provides an isolated nucleic acid that encodes an isolated protein according to the first aspect.

In particular embodiments, the isolated nucleic acid comprises a nucleotide sequence selected from the group consisting of: SEQ ID NO: 25; SEQ ID NO:26; SEQ ID NO: 27; SEQ ID NO: 28; SEQ ID NO: 29; SEQ ID NO: 30; SEQ ID NO: 31; SEQ ID NO:32; SEQ ID NO: 33; SEQ ID NO: 34; SEQ ID NO: 35; SEQ ID NO: 36; SEQ ID NO: 37; SEQ ID NO: 38; SEQ NO:39; SEQ ID NO: 40; SEQ ID NO: 41; SEQ ID NO: 42; SEQ ID NO: 43; SEQ ID NO: 44; SEQ NO:45; SEQ ID NO: 46; SEQ ID NO: 47; and SEQ ID NO: 48.

In a third aspect, the invention provides an immunotherapeutic composition comprising an isolated hypoxanthine guanine xanthine phosphoribosyl transferase (HGXPRT) protein, or at least one immunogenic fragment thereof, and an immunologically-acceptable carrier, diluent or excipient.

In a fourth aspect, the invention provides an immunotherapeutic composition comprising an isolated nucleic acid encoding hypoxanthine guanine xanthine phosphoribosyl transferase (HGXPRT) protein, or at least one immunogenic fragment thereof, and an immunologically-acceptable carrier, diluent or excipient.

Preferably, the immunotherapeutic composition is a vaccine.

Preferably, the HGXPRT protein, immunogenic fragment thereof or encoding nucleic acid is isolated or otherwise derived from a disease-causing protozoan.

Preferably, the HGXPRT protein, the or each immunogenic fragment thereof or encoding nucleic acid is isolated from a Plasmodium species or a Babesia species.

In one embodiment, said immunotherapeutic composition further comprises one or more B-lymphocyte epitopes, or a nucleic acid encoding same, obtained from one or more proteins other than HGXPRT.

In a fifth aspect, the invention provides an isolated lymphocyte that recognizes a HGXPRT protein epitope.

Preferably, the lymphocyte is a T-lymphocyte.

More preferably, the T-lymphocyte is a CD4⁺ T lymphocyte.

Preferably, the T-lymphocyte is a CD4⁺ T lymphocyte that produces one or more cytokines including but not limited to interleukin-2 (IL-2), interferon-gamma (IFN-γ) and tumour necrosis factor alpha (TNF-α).

In a sixth aspect, the invention provides a non-human animal immunized with a HGXPRT protein or at least one immunogenic fragment thereof.

Preferably, the non-human mammal is a mouse, cow, dog or chicken.

In a seventh aspect, the invention provides a method of immunization against a protozoal disease, said method including the step of administering a HGXPRT protein, or at least one immunogenic fragment thereof, to an animal.

In an eighth aspect, the invention provides a method of immunization against a protozoal disease, said method including the step of administering an isolated nucleic acid encoding a HGXPRT protein or an immunogenic fragment thereof, to an animal.

In a ninth aspect, the invention provides an antibody that binds an immunogenic fragment of a HGXPRT protein.

Preferably, the antibody binds a peptide comprising an amino acid sequence set forth in any one of SEQ ID NOS: 1-24.

It will be appreciated from the foregoing that the invention relates to immunotherapy using HGXPRT protein as immunogen treat any of a variety of protozoal diseases including, but not limited to, malaria in humans (Plasmodium spp.), Babesis bovis, Babesia bigemina and Babesia divergens infecting cattle, Babesia canis infecting dogs, Babesia microti and Babesia divergens infecting humans, Trypanosomiasis in man and animals, especially cattle (Trypanosoma gambiense, T congolense, T. cruzi and other species), the various forms of Leishmaniasis (Leishmania donovani, L. tropica, L. brasiliensis, L. mexicana) Giardiasis (Giardia lamblia), Toxoplasmosis (Toxoplasma gondii) and coccidiosis in chickens (Eimeria tenella) and Anaplasmosis.

Throughout this specification, unless otherwise indicated, “comprise”, “comprises” and “comprising” are used inclusively rather than exclusively, so that a stated integer or group of integers may include one or more other non-stated integers or groups of integers.

### BRIEF DESCRIPTION OF THE FIGURES

FIG. 1A Specificity of T-cell lines. Proliferative responses of T-cell lines to different antigens. The results are Δ CPM means of triplicate wells±SD. The background CPM for F-, J-, pRBC-, E- and ovalbumin-specific T cell lines were 2630±51, 560±83, 2573±325, 3330±403 and 274±46 respectively.

FIG. 1B Immunophenotypic and cytokine characterization of parasite-specific and ovalbumin T-cell lines. For each cell line, the proportion of cells is a representative result of cells from two 24-well plates.

FIG. 2 Parasitaemia levels of SCID mice (5 mice/group). The mice were transfused with 5×10⁶ antigen specific T-cells. One day later the mice were subjected to two cycles of infection-cure regimen before an i.v challenge infection with 10⁵ pRBC.

FIG. 3 Purification of F-pool of protein fractions to single band proteins. Preparative SDS-PAGE gradient gel (6-18%) stained with Coomassie blue to show single band proteins excised for analysis. The relative molecular weights of protein markers are indicated on the left. The arrows indicate the position of the doublet band 16.

FIG. 4A Parasitaemia levels of SCID mice to assay the single band proteins. The mice were adoptively transfused with single band specific T-cells (5×10⁶/mouse) that had undergone a single in vitro stimulation-rest culture cycle using F-fraction antigen. 24 hours following transfer, the cells were expanded in vivo by two cycles of infection-cure regimen before the mice were challenged i.v with parasitized erythrocytes (10⁵ pRBC/mouse).

FIG. 4B Parasitaemia levels in SCID mice to assay the single band proteins. The mice were transfused with F-antigen specific T-cells (10⁶/mouse) then immunized with single band proteins in CFA/IFA (total 5 μg/mouse) before undergoing two cycles of infection-cure regimen. They were then challenged i.v with parasitized erythrocytes (10⁵ pRBC/mouse).

FIG. 5A Proliferative responses of protective P. yoelii 17XNL band 16 (P1)— primed lymph node cells to the active form of recombinant P. falciparum HGXPRT (PfX antigen). Mice were immunized with the protective SDS-PAGE gel extracted band 16 (P1). Ten days later the lymph node cells were collected and stimulated with the band just below P1 (P2) (FIG. 3) and the PfX. An unrelated 100 kDa parasite protein (A) was used to test specificity. Whole P. yoelii parasite antigens (pRBC), PPD and con-A were used as controls. The results are mean CPM of triplicate wells ±SD. The background CPM was 2551±396.

FIG. 5B Parasitaemia levels in BALB/c mice immunized with P. falciparum HGXPRT (PfHGXPRT). Mice immunized with ovalbumin and PBS are included as controls.

FIG. 6 Alignment of HGXPRT sequences from Plasmodium species, humans and mice, and peptides CS 17 and CS21. Dark grey shading indicates identities; light shading indicates conservative substitutions. The predicted immunogenic amino acids of CS 17 and CS 21 are as indicated in bold.

FIG. 7(A) Peptide sequences (designated CS-1 to CS-24) from HGXPRT (derived from P. falciparum strain FCR3, and P. falciparum strain 3D7) and (3) nucleotide sequences encoding peptides CS-1 to CS-24. Peptides designated CS-1 to CS-24 respectively correspond to SEQ ID NOS:1-24. Nucleotide sequences encoding CS-1 to CS-24 respectively correspond to SEQ ID NOS:25-48.

FIG. 8 Proliferative responses of lymph node cells obtained from BALB/c mice immunized with recombinant PfHGXPRT (rPfHGXPRT). The results are means of triplicate wells. Lymph node T cells were stimulated in vitro either with peptides derived from rPfHGXPRT (designated CS-1 to CS-24), or the full length recombinant HGXPRT molecule (rPfHGXPRT). T cells were also stimulated with human or mouse red blood cells infected with various Plasmodium strains. T cells stimulated with purified protein derivative (PPD), normal mouse red blood cells (NMRBC) and normal human red blood cells (NHRBC) serve as controls. Key: rPfHGXPRT; recombinant Plasmodium falciparum HGXPRT; Pf-pRBC (3d7); human red blood cells infected with P. falciparum, strain 3d7; Pb-pRBC; mouse red blood cells infected with P. berghei, strain ANKA; P.ch AS-pRBC; mouse red blood cells infected with P. chabaudi, strain AS; Py 17XNL-pRBC; mouse red blood cells infected with P. yoelii, strain17XNL; Py YM-pRBC; mouse red blood cells infected with P. yoelii, strain YM; Pv-pRBC; mouse red blood cells infected with P. vinckei.

FIG. 9 The proliferative responses of lymph node cells from B10.BR mice immunized with either A. pooled peptides CS 1-8; B. pooled peptides CS 9-16 or C. pooled peptides CS 17-24. The lymph node cells were stimulated in vitro with either rPfHGXPRT, PPD, ConA (as a control), or varying concentrations (30, 10 and 3 ug/ml) of individual peptides, as indicated.

FIG. 10 Proliferative responses of lymph node cells from B10.BR mice immunised with A. pool of peptides CS 1-8, B. pool of peptides CS 9-16 or C. pool of peptides CS 17-24 when stimulated in vitro with red blood cells infected with different strains of Plasmodium. Refer to key in FIG. 3.

FIG. 11 The ability of rPfHIGXPRT peptide specific immune responses to protect B10.BR mice from infection with P. berghei ANKA. B10.BR mice were immunised with either peptide 16, 17, 21, pooled peptides 16, 17 and 21 or phosphate buffered saline (PBS; control) and then challenged with P. berghei ANKA.

FIG. 12 The proliferative responses of lymph node cells from BALB/c mice immunized with either A. pooled peptides CS1-8; B. pooled peptides CS9-16 or C. pooled peptides CS 17-24. The lymph node cells were stimulated in vitro with either PPD, ConA (as controls), or varying concentrations (30, 10 and 3 ug/ml) of the individual peptides from the pool of peptides that the mice were immunised with.

FIG. 13 Proliferative responses of lymph node cells from BALB/c mice immunised with A. pooled peptides CS 1-8, B. pooled peptides CS9-16 or C. pooled peptides CS17-24 when stimulated in vitro with red blood cells infected with different strains of Plasmodium.

FIG. 14 The ability of rPfHGXPRT peptide specific immune responses to protect BALB/c mice from infection with P. yoelii 17XNL. BALB/c mice were immunised with either peptide 1, 17, 21, pools of peptides 9, 10 and 11, pool of peptides 1, 9, 10, 11, 17 and 21 or PBS and then challenged with P. yoelii 17XNL.

FIG. 15 The ability of rPfHGXPRT peptide specific immune responses to protect BALB/c mice from infection with P. berghei ANKA. BALB/c mice were immunised with either peptides 1, 17, 21, pooled peptides 9, 10 and 11, pooled peptides 1, 9, 10, 11, 17 and 21 or PBS (control) and then challenged with P. berghei ANKA.

### DETAILED DESCRIPTION OF THE INVENTION

The present invention arises from the unexpected discovery that HGXPRT isolated from malaria parasites induces an immune response when administered to a mouse. Even more remarkably, protection mediated by the immune response is primarily a result of T-cells and may involve production of “Th1” cytokines such as interleukin-2 (IL-2), interferon-gamma (IFN-γ) and tumour necrosis factor alpha (TNF-α). Furthermore, the immune response is protective as judged by the ability of immunized mice to resist subsequent challenge with malaria parasites. The present invention also provides defined, immunogenic peptides derived from malarial HGXPRT that may be useful in inducing protective immune responses. This invention therefore logically extends to immunization of animals against any of a variety of protozoal diseases. In particular, the invention provides immunization of humans against malaria with an immunogen that may be the first, effective inducer of protective T cell-mediated immunity in humans.

Immunogenic Fragments of HGXPRT

In one aspect the invention provides isolated, immunogenic fragments of protozoal HGXPRT and isolated proteins comprising one or more of said immunogenic fragments.

For the purposes of this invention, by “isolated” is meant material that has been removed from its natural state or otherwise been subjected to human manipulation. Isolated material may be substantially or essentially free from components that normally accompany it in its natural state, or may be manipulated so as to be in an artificial state together with components that normally accompany it in its natural state. Isolated material may be in native or recombinant form.

By “protein” is meant an amino acid polymer. The amino acids may be natural or non-natural amino acids, D- or L-amino acids as are well understood in the art. Also included within the scope of amino acids are chemically-modified or derivatized amino acids as are well known in the art.

A “peptide” is a protein having less than fifty (50) amino acids.

A “polypeptide” is a protein having fifty (50) or more amino acids.

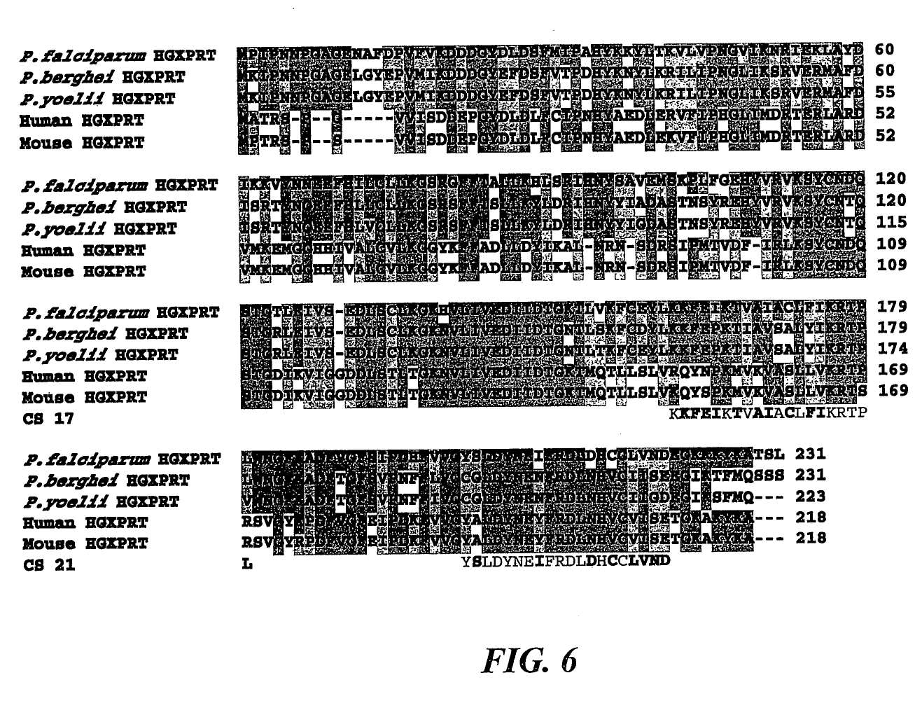

HGXPRT proteins isolated from malaria parasites are well known in the art and are readily available to the skilled person. HGXPRT protein sequences from Plasmodium species, mouse and human are shown in FIG. 6.

Hypoxanthine guanine xanthine phosphoribosyl transferase (HGXPRT) is common in mammalian, protozoal and bacterial species. For example, BLAST sequence searches of available databases shows that HGXPRT is identifiable in Leishmania major, Leishmania donovani, Trypanosoma cruzi, Trypanosoma brucei, Toxoplasma gondii and Eimeria tenella. For example, the enzyme in T. gondii shows 51% sequence identity and 70% amino acid similarity to that of Plasmodium falciparum, while the partial amino acid sequence of E. tenella shows 44% sequence identity and 65% sequence similarity to the enzyme from P. falciparum.

It should also be noted that xanthine is an additional substrate used by Plasmodium, Toxoplasma and Trichomonas HGPRT enzymes, whose enzymes are therefore abbreviated on occasion as HGXPRT. This represents a minor difference in substrate specificity that is independent of immunological response to the antigen. For the purposes of this specification all hypoxanthine guanine xanthine phosphoribosyl transferase enzymes will be abbreviated as HGXPRT.

In the context of the present invention, “an immunogenic fragment” of a HGXPRT protein is any amino acid sequence present in a HGXPRT protein (other than the entire HGXPRT amino acid sequence) that is capable of eliciting an immune response when administered to an animal, preferably a mammal.

By “epitope” is meant a contiguous or non-contiguous sequence of amino acids that is recognized by at least one T cell or B cell clonotype in vivo or in vitro.

Isolated proteins of the invention may comprise at least one immunogenic fragment as described herein and, optionally, additional amino acid residues.

In light of the foregoing, it will be appreciated that the invention contemplates isolated proteins that comprise a plurality of immunogenic fragments as described herein and, optionally, additional amino acid residues.

The examples of isolated proteins set forth in SEQ ID NOS:1-24 comprise at least one amino acid sequence derived from or present in a Plasmodium HGXPRT protein, in particular embodiments together with additional amino acid residues. The at least one amino acid sequence has generally been selected by virtue of being a minimal region non-homologous to HGXPRT protein sequences in mammals such as mice and humans. These minimal, non-homologous regions may be the preferred targets of protective T cells.

Particularly efficacious isolated proteins of the invention are set forth in SEQ ID NOS: 6, 7, 8, 9, 10, 11, 12, 15, 17, 18, 21, 22, 23 and 24.

Even more efficacious isolated proteins of the invention are set forth in SEQ ID NOS: 17 and 21.

As shown in FIG. 6, the alignment of the immunogenic peptides CS 17 (SEQ ID NO:17 and CS 21 (SEQ ID NO:21) to highly homologous regions of the human and mouse HGXPRT proteins is a surprising result. It would have been expected that these immunogenic fragments would have aligned to non-homologous regions.

The predicted immunogenic amino acids of CS 17 and CS 21 are indicated in FIG. 6, although this should not be taken as an indication that these must be immunogenic residues, or that there are not other immunogenic residues in CS 17 and CS 21.

Identification of other T cell epitopes present in HGXPRT may be undertaken by the skilled person based on the enabling disclosure provided herein. In this regard, reference is made to Tian et al., 1998, which provides an example of epitope-mapping methodology that may be applicable to mapping HGXPRT T cell epitopes.

The invention also contemplates variants and/or derivatives or other modified HGXPRT proteins and immunogenic fragments that nevertheless retain immunogenicity. Indeed, it will be appreciated that SEQ ID NOS:1-24 are not entirely derived from a corresponding HGXPRT sequence, but instead include additional amino acids, and hence are “variants” with respect to the corresponding HGXPRT sequence.

Generally, conservative amino acid substitutions may be introduced or the HGXPRT protein that essentially retain immunogenicity. Alternatively, non-conservative amino acid substitutions may be introduced that modify immunogenicity as desired.

It will also be appreciated that computer-assisted analysis of T cell epitopes may be useful in creating variants of HGXPRT immunogenic fragments or peptides, using approaches such as described in Dressel et al., 1997, or in Michielin et al., 2000, although without limitation thereto.

In light of the foregoing, in particular embodiments variants of the immunogenic proteins of the invention may comprise an amino acid sequence having at least 70%, preferably at least 80%, more preferably at least 90% and advantageously at least 95% sequence identity with any one of SEQ ID NOS:1-24.

Sequence identity may be measured over a “comparison window” of at least six amino acids, preferably at least twelve amino acids, more preferably at least twenty amino acids and advantageously over substantially the entire length of a reference amino acid sequence.

It is also contemplated that HGXPRT protein and fragments thereof may be chemically cross-linked to a carrier protein (such as BSA or thyroglobulin). Other types of chemical modifications of particular amino acids are well known in the art, although the skilled person is referred to Chapter 15 of CURRENT PROTOCOLS IN PROTEIN SCIENCE Eds. Coligan et al. John Wiley & Sons NY USA (1995-2001) for more detailed methodology relating to chemical modification of proteins.

It will also be appreciated immunotherapeutic compositions according to the invention may also include one or more B-cell epitopes from one or more protozoan antigens other than HGXPRT. Examples include the carboxy terminus of MSP1 or Apical membrane Antigen 1 as B cell epitopes, although without limitation thereto.

It will be understood that the preferred immune response is a protective T lymphocyte-mediated response against malaria in humans.

HGXPRT proteins, immunogenic peptides derived therefrom and other protein or peptide components of vaccines (such as B-cell epitopes) may be produced by recombinant DNA technology or by solid or liquid phase chemical synthesis as are well known in the art.

Recombinant protein expression is well known in the art and expression systems are available in bacterial (e.g E. coli DH5 α), insect (e.g. Sf9) and yeast cells.

By way of example, the skilled person is referred to Chapters 5 and 18 of CURRENT PROTOCOLS IN PROTEIN SCIENCE Eds. Coligan et al. John Wiley & Sons NY USA (1995-2001) for techniques applicable to recombinant protein expression and chemical synthesis respectively.

Alternatively, peptides can be produced by digestion of a HGXPRT protein with proteinases such as endoLys-C, endoArg-C, endoGlu-C and staphylococcus V8-protease. The digested fragments can be purified by, for example, high performance liquid chromatographic (HPLC) techniques.

For the purposes of recombinant HGXPRT protein expression, a fusion partner may be added to assist purification. By way of example, fusion partners include a polyhistidine tag, maltose binding protein (MBP), Protein A and glutathione S-transferase (GST). It will also be appreciated that protease cleavage sites (eg. factor Xa and thrombin sites) may be present between the HGXPRT protein and fusion partner to allow subsequent removal of the fusion partner.

HGXPRT Nucleic Acids and Nucleic Acid Vaccines

In one form, the invention provides an isolated nucleic acid encoding one or more immunogenic fragments of a protozoan HGXPRT, preferably a malarial HGXPRT.

Malaria parasite HGXPRT nucleic acid sequences are well known in the art, although reference is made to NCBI Entrez accession numbers M88110 (Plasmodium falciparum) and AB021413 (Plasmodium berghei).

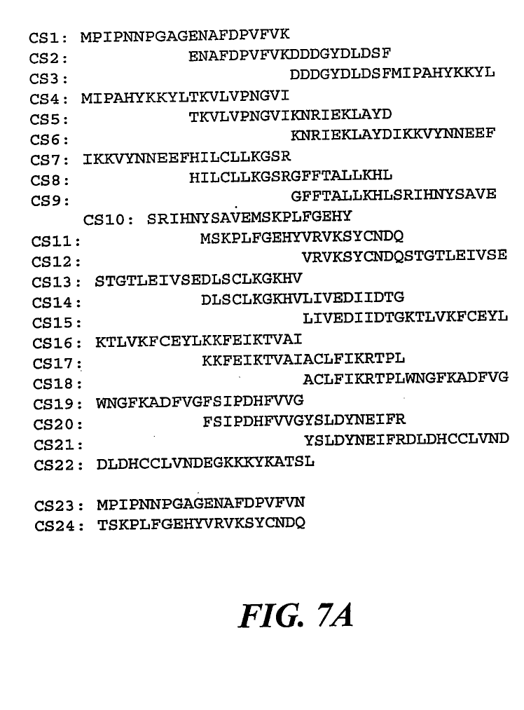

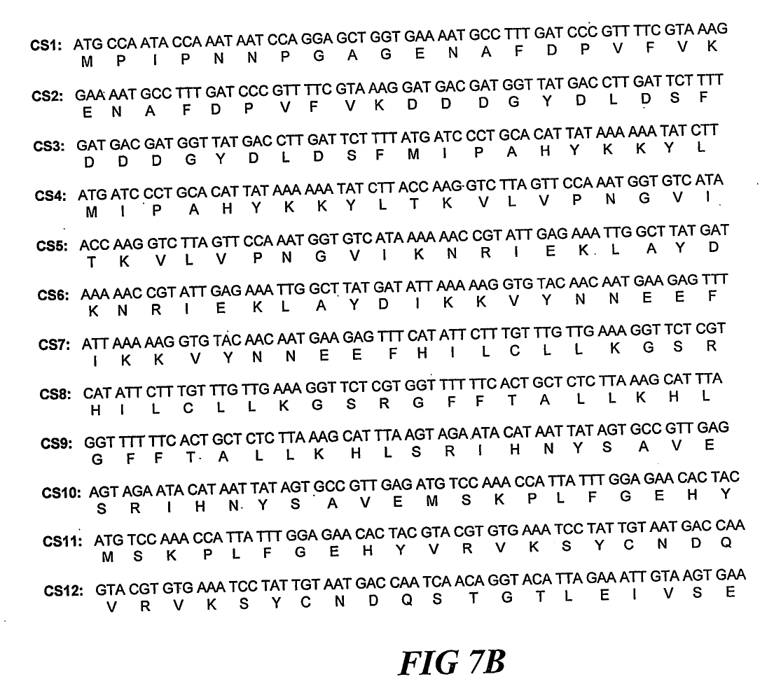

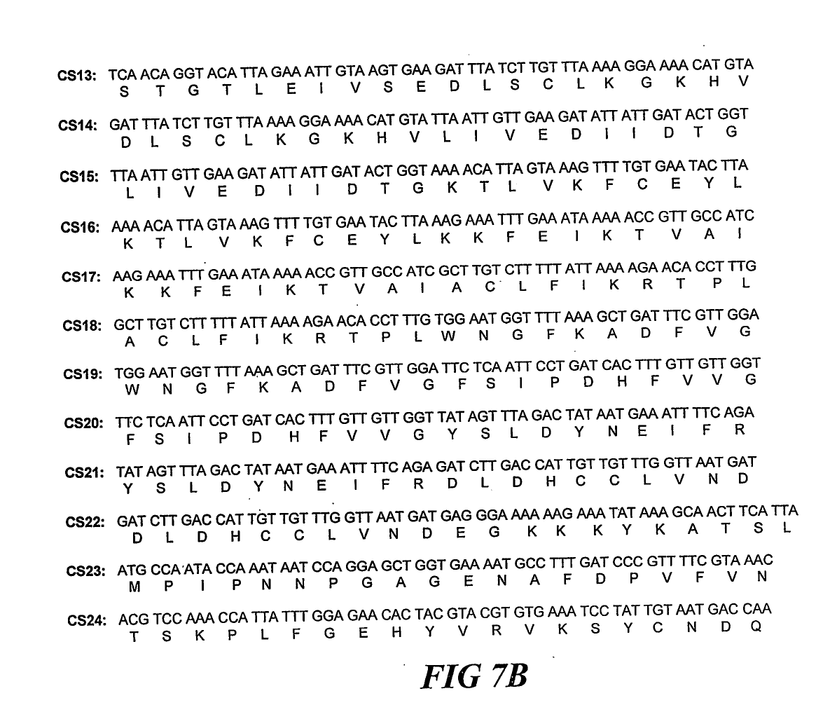

Preferred nucleic acid sequences encoding the peptides of SEQ ID NOS:1-24 are respectively set forth in SEQ ID NOS:2548 and in FIG. 7B.

The invention also provides an immunotherapeutic composition that includes a nucleic acid encoding HGXPRT or one or more immunogenic fragments thereof, preferably in the form of a DNA vaccine.

Said nucleic acid may also encode one or more B-cell epitopes from one or more malaria antigens other than HGXPRT.

Accordingly, DNA vaccines inclusive of polyepitope constructs are also contemplated by the present invention, using methodology such as described in Boyle et al., 1998 or in Hanke et al., 1999.

The term “nucleic acid” as used herein designates single-or double-stranded mRNA, RNA, cRNA and DNA, inclusive of cDNA, genomic DNA and DNA-RNA hybrids.

A “polynucleotide” is a nucleic acid having eighty (80) or more contiguous nucleotides, while an “oligonucleotide” has less than eighty (80) contiguous nucleotides.

A “probe” may be a single or double-stranded oligonucleotide or polynucleotide, suitably labeled for the purpose of detecting complementary sequences in Northern or Southern blotting, for example.

A “primer” is usually a single-stranded oligonucleotide, preferably having 15-50 contiguous nucleotides, which is capable of annealing to a complementary nucleic acid “template” and being extended in a template-dependent fashion by the action of a DNA polymerase such as Taq polymerase, RNA-dependent DNA polymerase or Sequenase™.

The invention also contemplates use of modified purines (for example, inosine, methylinosine and methyladenosine) and modified pyrimidines (thiouridine and methylcytosine) in nucleic acids of the invention.

The present invention also contemplates HGXPRT-encoding nucleic acids that have been modified such as by taking advantage of codon sequence redundancy. In a particular example, codon usage may modified to optimize expression of a nucleic acid in a particular organism or cell type. In this regard, it has been shown that in the context of DNA vaccines, codon optimization may enhance the immunogenicity of expressed malaria antigens (Narum et al., 2001).

The invention also contemplates modified nucleic acid that encode variant immunogenic fragments of HGXPRT as hereinbefore defined.

Modified nucleic acids may have at least 60%, preferably at least 70%, more preferably at least 80% and advantageously at least 90% nucleotide sequence identity with any one of SEQ ID NOS:2548, for example.

Sequence identity may be measured over a “comparison window” of at least twelve nucleotides, preferably at least twenty nucleotides, more preferably at least fifty nucleotides and advantageously over substantially the entire length of a reference nucleotide sequence.

For the purposes of a nucleic acid vaccine, an expression construct may be used which comprises an isolated HGXPRT-encoding nucleic acid operably linked to one or more regulatory sequences in an expression vector.

Regulatory nucleotide sequences present in the expression vector (such as an enhancer, promoter, splice donor/acceptor signals, terminator and polyadenylation sequences) are well known in the art facilitate expression of the chimeric receptor. Selectable markers are also useful whether for the purposes of selection of transformed bacteria (such as bla, kanR and tetR) or transformed mammalian cells (such as hygromycin, G418 and puromycin).

Both constitutive and inducible promoters may be useful for expression of chimeric receptors according to the invention. Examples of inducible promoters are metallothionine-inducible and tetracycline-repressible systems as are well known in the art.

Also contemplated are tissue-specific promoters that facilitate targeted expression of DNA vaccines to thereby enhance processing and presentation of the encoded immunogen.

It will be appreciated by the skilled person that DNA vaccination is becoming an increasingly used mode of vaccination against malaria. A full discussion of the expression vectors used and malaria antigens tested is beyond the scope of this specification. However, the skilled person is referred to Kumar et al., 2002, and Doolan & Hoffman, 2001, for a current overview of anti-malarial DNA vaccines.

Immunotherapeutic Compositions and Vaccines

The invention provides immunotherapeutic compositions, preferably vaccines, an immunogenic component of which is an isolated HGXPRT protein or one or more fragments thereof.

By “immunotherapeutic composition” is meant a composition that is capable of eliciting an immune response to one or more immunogenic components of the composition, or to an organism from which said one or more immunogenic components are derived.

As used herein, a “vaccine” is an immunotherapeutic composition that elicits a protective immune response.

It will be appreciated that immunotherapeutic compositions and vaccines may be used therapeutically to treat malaria or may be used prophylactically to prevent or reduce the severity of protozoan infestation.

Immunotherapeutic compositions of the invention inclusive of HGXPRT protein, peptide and nucleic acid-based therapeutics may be administered in any of a number of forms and routes of administration.

It is preferred that immunotherapeutic compositions, such as vaccines of the invention, include an immunologically-acceptable carrier, diluent or excipient, which in some cases may be an adjuvant. However, it will also be appreciated that an immunologically-acceptable carrier, diluent or excipient may be a substance such as water, saline, alcohol, an organic polymer or other immunologically-inert carrier that merely assists vaccine delivery by appropriately suspending and/or solubilizing vaccine components.

As will be understood in the art, an “adjuvant” means a composition comprised of one or more substances that enhances the immunogenicity and efficacy of a vaccine composition. Non-limiting examples of suitable adjuvants include squalane and squalene (or other oils of animal origin); block copolymers; detergents such as Tween®-80; Quil® A, mineral oils such as Drakeol or Marcol, vegetable oils such as peanut oil; Corynebacterium-derived adjuvants such as Corynebacterium parvum; Propionibacterium-derived adjuvants such as Propionibacterium acne; Mycobacterium bovis (Bacille Calmette and Guerin or BCG); interleukins such as interleukin 2 and interleukin 12; monokines such as interleukin 1; tumour necrosis factor; interferons such as gamma interferon; combinations such as saponin-aluminium hydroxide or Quil-A aluminium hydroxide; liposomes; ISCOM® and ISCOMATRIX® adjuvant; mycobacterial cell wall extract; synthetic glycopeptides such as muramyl dipeptides or other derivatives; Avridine; Lipid A derivatives; dextran sulfate; DEAE-Dextran or with aluminium phosphate; carboxypolymethylene such as Carbopol EMA; acrylic copolymer emulsions such as Neocryl A640 (e.g. U.S. Pat. No. 5,047,238); vaccinia or animal poxvirus proteins; sub-viral particle adjuvants such as cholera toxin, or mixtures thereof.

Any safe route of administration may be employed for administering an immunotherapeutic composition of the invention. For example, oral, rectal, parenteral, sublingual, buccal, intravenous, intra-articular, intra-muscular, intra-dermal, subcutaneous, inhalational, intraocular, intraperitoneal, intracerebroventricular, transdermal and the like may be employed. Intra-muscular and subcutaneous injection is particularly appropriate, for example, for administration of immunotherapeutic compositions such as protein-based and DNA-based vaccines.

Antibodies

The invention also provides antibodies that bind immunogenic fragments of HGXPRT, such as the peptide sequences set forth in any on of SEQ ID NOS:1-24, or variants thereof.

Antibodies of the invention may be polyclonal or monoclonal. Well-known protocols applicable to antibody production, purification and use may be found, for example, in Chapter 2 of Coligan et al., CURRENT PROTOCOLS IN IMMUNOLOGY (John Wiley & Sons NY, 1991-1994) and Harlow, E. & Lane, D. Antibodies: A Laboratory Manual, Cold Spring Harbor, Cold Spring Harbor Laboratory, 1988.

In particular, monoclonal antibodies may be produced using the standard method as for example, described in an article by Köhler & Milstein, 1975, Nature 256, 495, which is herein incorporated by reference, or by more recent modifications thereof as for example, described in Coligan et al., CURRENT PROTOCOLS IN IMMUNOLOGY, supra by immortalizing spleen or other antibody producing cells derived from a production species which has been inoculated with one or more of the polypeptides, fragments, variants or derivatives of the invention.

The invention also includes within its scope antibodies which comprise Fc or Fab fragments of the polyclonal or monoclonal antibodies referred to above. Alternatively, the antibodies may comprise single chain Fv antibodies (scFvs) against the BSP proteins of the invention. Such scFvs may be prepared, for example, in accordance with the methods described respectively in U.S. Pat. No. 5,091,513, European Patent No 239,400 or the article by Winter & Milstein, 1991, Nature 349 293, which are incorporated herein by reference.

Labels may be associated with an antibody of the invention, or antibody fragment, and may be selected from a group including a chromogen, a catalyst, an enzyme, a fluorophore, a chemiluminescent molecule, a lanthanide ion such as Europium (EU³⁴), a radioisotope and a direct visual label. In the case of a direct visual label, use may be made of a colloidal metallic or non-metallic particle, a dye particle, an enzyme or a substrate, an organic polymer, a latex particle, a liposome, or other vesicle containing a signal producing substance and the like.

A large number of enzymes useful as labels is disclosed in United States Patent Specifications U.S. Pat. No. 4,366,241, U.S. Pat. No. 4,843,000, and U.S. Pat. No. 4,849,338, all of which are herein incorporated by reference. Enzyme labels useful in the present invention include, for example, alkaline phosphatase, horseradish peroxidase, luciferase, β-galactosidase, glucose oxidase, lysozyme, malate dehydrogenase and the like. The enzyme label may be used alone or in combination with a second enzyme in solution.

The fluorophore may, for example, be fluorescein isothiocyanate (FITC), tetramethylrhodamine isothiocyanate (TRITL), allophycocyanin (APC), Texas Red, Cy5, Cy3, or R-Phycoerythrin (RPE) as are well known in the art.

So that the invention may be understood in more detail, the skilled person is referred to the following non-limiting examples.

### EXAMPLES

### Example 1

### Identification of HXGPRT as an Immunogen in Plasmodium yoelii

1. Materials and Methods

Mice

Four to six week old normal BALB/c, athymic BALB/c nude, and BALB/c SCID mice were purchased from the Animal Resources Center (Perth, WA, Australia) and housed at the animal facility of QIMR under specific-pathogen-free conditions. They were used in experiments when they were 6-8 weeks old.

Parasites

The rodent malaria parasites, P. yoelii 17×NL was maintained by alternating passage of 10⁶ parasitized red blood cells (pRBC) per mouse between infected and uninfected mice via the intraperitonial route (i.p). After three or four passages the stabilized parasites were then frozen and stored in liquid nitrogen. Three alternate passages were performed before cryopreservates were used for experimental challenge infections. Parasites were collected from blood obtained by cardiac puncture and tail snip and used to prepare parasite antigens and thin blood smear to determine parasitaemia respectively.

Determination of Parasitaemia

A thin blood smear on a glass slide was made from a drop of blood collected from an infected mouse by tail snip. The smear was air-dried and stained using Diff-Quick staining reagents (Lab Aids Pty Ltd, Narrabeen, Australia). About 300-500 total red blood cells (RBC) were counted to estimate percent parasitaemia; however at least 10³ RBCs were enumerated to determine parasitaemia of less than or equal to 1%.

Preparation of Parasite Antigens

Preparation of Whole Parasite Antigen (pRBC)

Blood was collected from infected mouse when the parasitaemia was between 30-50% by heart puncture into heparinized sterile tube. The blood was then washed in warm PBS and the concentration of pRBC was determined. The pRBC in PBS was then used immediately for infecting mice or pRBC lysed by incubation in erythrocyte lysis buffer (0.17M tris-hydroxymethyl aminomethane, 0.16M ammonium chloride; pH 7.2) at 37° C. for 10 mins. The parasites were then freeze-thawed at least three times, sonicated, aliquoted and stored at −70° C. to be used for in vitro cell cultures.

Preparation of Soluble Parasite Antigen (sAg)

Blood was collected when parasitaemia was about 50-60% by heart puncture into heparinized tubes. This was then washed twice in PBS. RBCs were lysed by incubation of the blood in 0.01% saponin/PBS at 37° C. for 20 mins in the presence of protease inhibitors. The blood was then given a further wash in saponin/PBS buffer before the pRBCs were sonicated in cold PBS at 4° C.

Following sonication, the lysate was centrifuged at 100,000 xg for 30 min at 4° C. The supernatant was then collected and dialyzed against three changes of PBS at 4° C. The protein concentration was determined by bicinchoninic acid assay and the soluble parasite antigen stored at −70° C. until use.

Generation of T cell Lines in vitro

Mice were immunized s.c at the hind footpads with 50 ul of antigen emulsified in complete Freund's adjuvant (CFA) (Sigma, St. Louis, Mo., USA). 7-10 days later, inguinal and popliteal lymph nodes were collected into Eagle's minimal. essential medium with Earle's Salts (MEM) (Trace, Biosciences, Australia) under aseptic conditions. Lines were then produced as previously described (Amante & Good, 1997) using alternate cycles of stimulation and rest.

Lymphocyte Proliferation Assay

A suspension of T-cells taken after rest phase and mixed with irradiated syngeneic splenocytes (1:3) containing no RBCs, were cultured at 2×10⁶ cells/ml complete culture medium for 4 days in 96-flat-well microtitre plates (Corning Incorporated, Corning) in humidified incubator containing 5% CO₂ at 37° C. Cells were in triplicate or quadruplicate wells either in medium only (negative control), tuberculin purified protein derivative (PPD) (CSL Ltd, Parkville, VIC, Australia) or concanavalin-A (con-A) as positive controls or with varying concentrations of test antigen(s), for the assessment of proliferation and/or the level of cytokines in culture supernatants at 24, 48 and/or 72 hours.

Proliferation was measured by pulsing for the last 16-18 hours of culture with 0.25 uCi/well ³H-thymidine (Amersham Biotech, UK) and harvesting the contents of each well onto glass fibre mats. The mats were air-dried and the incorporation of ³H-TdR determined using a plate scintillation counter.

Adoptive Transfer and Infection-Cure Regimens

Viable T cells harvested at resting phase were purified by centrifugation over Ficoll-Hypaque (Pharmacia). After washing in EMEM at 300×g RT the cells were resuspended in PBS and 200 ul containing appropriate number of cells transfused i.v into each immunodeficient mouse via the lateral tail vein. 24 hrs following transfusion, the T cells were expanded in vivo by an i.v infection of 10⁵ pRBC/mouse. Two days later the mice were treated i.p with pyrimethamine (Dosage: 0.2 mg/0.2 ml PBS/mouse/day) for 3 days. This cycle of infection-cure regimen was repeated before mice were given a challenge infection (10⁵ pRBC/mouse) and then followed for parasitaemia and sera. The mice were allowed three-week intervals between reinfections to allow for complete drug metabolism.

Cytokine ELISA

Supernatants from cell cultures were collected and assayed immediately or aliquoted and stored frozen at −70° C. until use. IL-4 and IFN- were measured by enzyme-linked immunosorbent assay (ELISA) based on the protocol of Sander et al. (1993), using Mab clone BVD4-1 D111 (5 ug/ml) and R4-6 A2 (2 ug/ml) (PharMingen, San Diego, USA) respectively, in bicarbonate coating buffer (pH 9.6) for coating 96-U-well plates (Immulon 2 HB; Dynatech Laboratories, USA) at 37° C. for 2 hrs. Undiluted supernatants were then serially titrated, plates wrapped in aluminium foil and incubated overnight in humidified chamber at room temperature (RT).

The plates were washed six times each successively in 0.05% Tween-20/PBS, PBS alone and water, and patted dry. Blocking buffer (0.05% Tween-20, 1% FCS, 0.1% skim milk powder in PBS) containing biotinylated BVD6-24G2 clone anti-mouse IL-4 (1:2000) or XMG1.2 rat anti-mouse IFN (1.0 ug/ml) (PharMingen) Mabs was added and the plates incubated for 2 hrs, RTP. After washes, streptavidin-HRP-conjugate (Vector, Burlingame, Calif., USA) was added (1:1600) for detection followed by a further 2 hrs, RT incubation before addition of the enzyme substrate substrate, 2,2-azinobis 3-ethylbenzthiazoline-6-sulphonic acid (ABTS) (Sigma). The absorbance was read at 415 nm with a reference of 490 nm after 1 hr. Recombinant cytokines (IL-4 and IFN; Sigma) and complete culture medium were assayed as positive and negative references respectively. Positive supernatants were defined as those that exceeded by at least 3SD the value for negative reference. Positive references were used to establish titration curves.

Intracellular Cytokine Staining

Viable rested T cell lines were purified by centrifugation over Ficoll Paque (Pharmacia Biotech) and resuspended in complete culture medium. They were then stimulated at 37° C. in humidified incubator containing 5% CO₂ for 6 hrs, with 40 ng/ml phorbol myristate acetate (PMA) (Sigma) and 2 uM calcium-ionophore (Sigma) in the presence of monensin (GolgiStop™, PharMingen) in accordance to manufacturer's instructions. Cells were washed twice in FACS buffer (0.1% BSA, 0.1% sodium azide/PBS) at 300 xg, 4° C. for 5 min and stained with a 1:50 FITC-conjugated rat anti-mouse CD4 monoclonal antibodies (Mabs) (Caltag Laboratories, Burlingame, Calif., USA) at 2×10⁷cells/ml FACS buffer on ice for 30 min in the dark. After two washes cells were thoroughly resuspended and fixed in 4% ice-cold paraformaldehyde at 4° C. for 30 min. Following two washes the cells were further fixed and permeabilized by incubation in cytofix/cytosperm™ (PharMingen) for 20-30 min at 4° C. according to the manufacturer's instructions. The cells were washed twice in permeabilization buffer, resuspended at 2×10⁷cells/ml cytofix/cytosperm™ and approximately 10⁶ cells dispensed into FACS tubes. The cells were then stained for 30 min at 4° C. in the dark, for IL-2, IFN-γ, TNF-α and IL-4 using a 1:50 PE-conjugated rat anti-mouse IL-2, IFN-γ, TNF-α and IL-4 monoclonal Mabs (PharMingen) respectively. As controls, unstained cell samples and PE-labelled nonspecific IgG1 and IgG2b Mabs (PharMingen) were used. After two further washes in permeabilization buffer cells were resuspended in 200 ul FACS buffer for immediate flow cytometric evaluation.

Fluorescence was analyzed using a FACScalibur flow cytometer (Becton Dickinson, Calif., USA) equipped with CELLQuest™ software. For each cytokine, 10,000 events were counted and the proportion of positive cells determined after correction for autofluorescence and non-specific fluorescence.

Analysis of Cell Surface Phenotypes

Cells were stained for surface expression of the following molecules: CD3, CD4, CD8, CD25, CD19, NK1.1, TCR and/or TCR, essentially as previously described (Xu et al, 2000).

Immunization and Challenge Infections

Groups of 3-5 mice were immunized s.c on the hind footpad with antigen emulsified in complete Freund's adjuvant (CFA) (Sigma). At four and six weeks later, booster immunizations were given s.c on the abdomen and i.p respectively using same amount of antigen emulsified in incomplete Freund's adjuvant (IFA) (Sigma). PBS mixed with adjuvant was used as control antigen.

10 days after the last booster the mice were challenged i.v with P. yoelii 17XNL parasitized RBC.

Serum Analysis by ELISA for Parasite-specific Antibodies

This was performed as previously described (Xu et al, 2000).

Coomassie Staining of Proteins in Gels

Proteins separated by SDS-PAGE were stained by incubation of the gel in 0.5% w/v Coomassie blue solution (containing 25% v/v methanol, 10% v/v acetic acid) for 1 hour with gentle rocking. The gel was then destained in three changes of destainer solution (a solution with similar composition but without the Coomassie blue stain) overnight.

Sodium Dodecyl Sulphate Polyacrylamide Gel Electrophoresis (SDS-PAGE)

A standard method described by Laemmli (1970) was used with few modifications. The gel was cast using acrylamide:bis-acrylamide (29:1).

Protein samples were mixed (4:1) with sample buffer (200 mM Tris, pH6.8; 30% v/v glycerol, 6% w/v SDS, 0.2% w/v bromophenyl blue) and incubated at 95° C. for 5 min prior to loading. Where protein reduction was necessary, β-mercaptoethanol or dithiothreitol (DTT) was added to the sample buffer. After loading, the gel was electrophoresed at constant voltage (60V) for approximately 1 hr 30 min. The proteins were then visualized by staining with Coomassie brilliant blue R-250 (Sigma) or silver stain by the method of Rabilloud et al (1988).

Preparative Isoelectric Focusing (IEF) for Fractionation of Soluble Parasite Proteins

Preparative wide-range (WR) and narrow-range (NR) isoelectric focusing (EF) was performed on a Multiphor II™ (Amersham Pharmacia Biotech, Uppsala) according to the manufacturers instructions. Briefly, the Zwittergent®3-12 soluble material was separated on wide-range isoelectric focusing (WR-IEF) according to the manufacturer's instructions. Briefly, 18 ml of Ampholine™ pH 3.5-10.0 (Amersham Pharmacis Biotech, Uppsala) and 1.35 g Zwittergent® 3-12 were mixed with MQW to a final volume of 16.0 g Ultrodex™ granulated gel (Amersham Pharmacia Biotech, Uppsala) added gradually and allowed to swell fu lly. A flat bed gel was prepared (19 cm×24 cm×0.5 cm deep) and the IEF gel pre-focused at 12 W and 10° C. for 1500 Vh. To 19.0 ml of the Zwittergent® 3-12 soluble material, 1.36 ml Ampholine™ pH 3.5-10.0 and approximately 1.0 g Ultrodex™ granulated gel were combined. This sample was loaded into the prefocused IEF gel at the low pH end (region of final pH ˜4.0-4.3) and focused at 12 W and 10° C. for 1200 Vh. Following electrophoresis, fractions were eluted with 2×2 ml MQW containing 0.2% Zwittergent® 3-12 and 50 μM AEBSF. The pH of each fraction was recorded and the sample pH adjusted to 7.4 with 1.0 ml of 1.0M Tris-HCl pH-7.4 buffer. The protein components of the IEF fractions were visualised on SDS-PAGE.

Passive Elution of Proteins from SDS-PAGE Gels

Coomassie stained gels were washed in water for 30 min. The gel was laid on a glass plate and using a sterile scalpel single bands excised into small cubes which were then placed into tubes containing extraction buffer (100 mM sodium acetate, 0.1% SDS, 10 mM DTT) and incubated overnight at 37° C. The supernatants were collected and analyzed by SDS-PAGE to confirm purity and subsequently transferred onto PVDF membrane.

PVDF-Membrane Electroblotting and N-terminal Sequencing

Proteins were separated on SDS-PAGE and blotted onto PVDF membrane using the electrophoresis unit, Multiphor II™ (Amersham Pharmacia Biotech, Uppsala, Sweden) by passage of 225 mAmps (1 mAmp/cm²) through blotting papers previously soaked in blotting buffer (0.02% w/v SDS, 0.3% w/v glycine, 0.15% v/v ethanolamine) for 1¼ hrs. The membrane was then rinsed in water and then methanol before being stained with coomassie blue (0.05% w/v coomassie blue, 40% v/v methanol, 1% v/v acetic acid) for 5 min. PVDF membrane was then destained in three changes of 50% v/v methanol each for 5 mins before two rinses in water. The membrane was placed at 37° C. incubator to dry and the band of interest excised and sequenced sequencing by Edman degradation method (Edman and Begg, 1967) on an N-terminal amino acid sequencer (Procise™; Applied Biosystems, Fostercity, Calif., USA).

2. Results

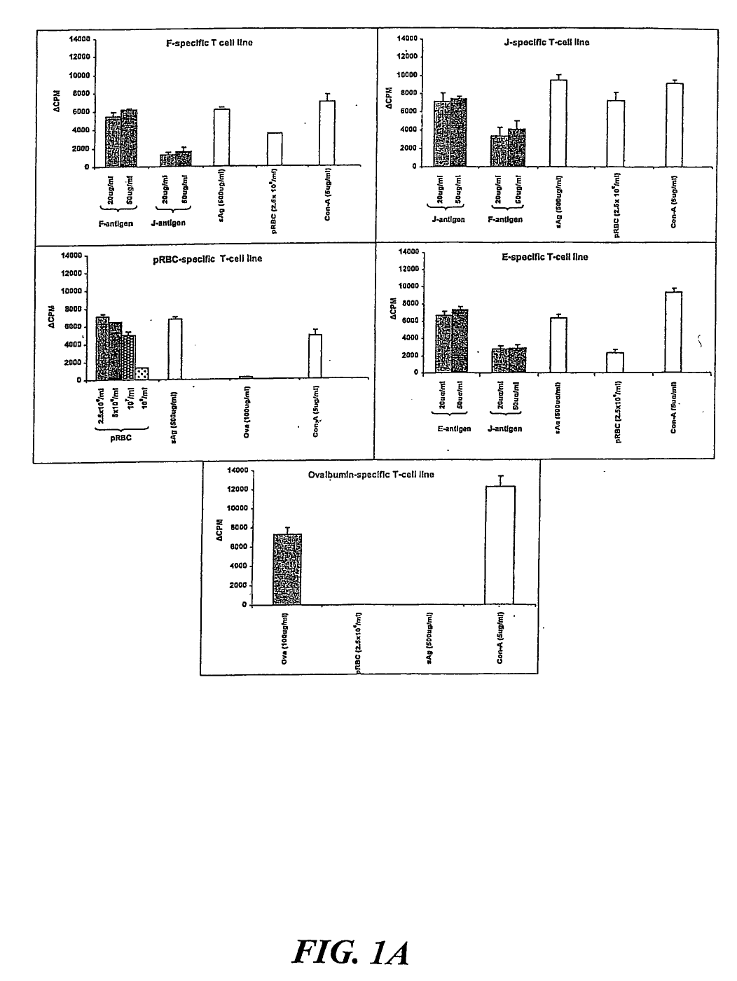

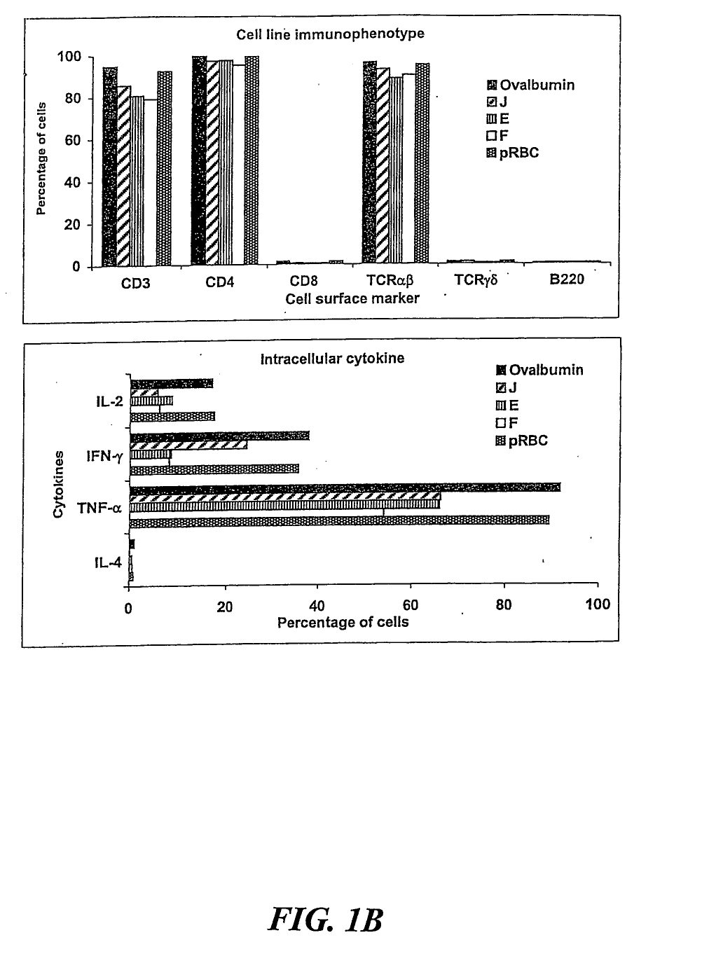

A soluble preparation of P. yoelii infected RBCs was made and this was then fractionated using charge into 30 fractions that were grouped into 12 pools (A-L). These individual fractions were then emulsified in complete Freund's adjuvant and used to immunize mice. Draining lymph nodes were taken 8 days later and following successive cycles of antigenic stimulation and rest, polyclonal T cell lines were produced. Lines were successfully established for only E, F and J fractions. The lines were tested for their ability to respond to the individual fractions, to the soluble parasite preparation and to parasitized RBCs. The lines demonstrated antigenic specificity (FIG. 1A) and expressed CD3, CD4 and αβ T cell receptors and produced cytokines (IL2, IFN-γ, TNF-α, but not IL4) consistent with them being “Th1” or pro-inflammatory cells (FIG. 1B). The lines were then administered to athymic nude BALB/c mice which were then challenged with a live parasite infection. On the basis of these results, one line, which demonstrated the most potent anti-parasite activity, was chosen for further investigation (fraction F line). It was noted, however, that recipients of Fraction F-specific T cells died (at low parasitemia) following challenge, as did the recipients of the other lines.

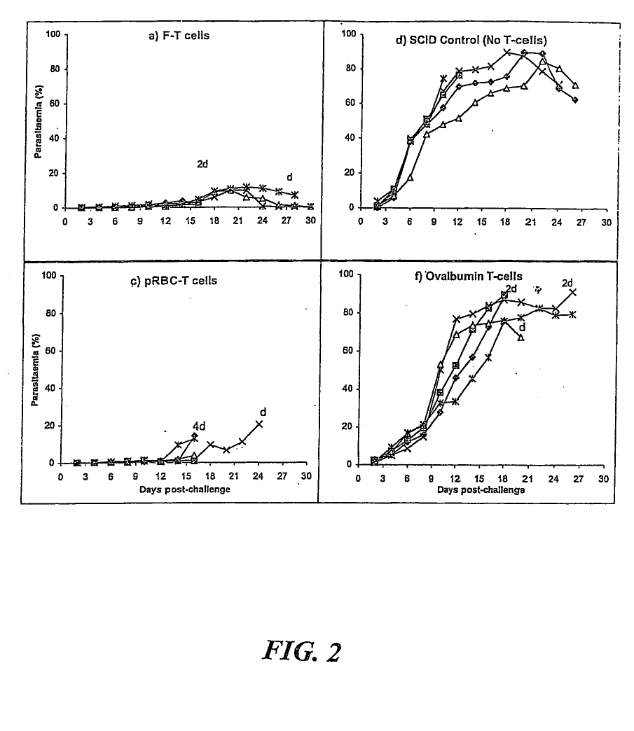

The testing procedure was therefore modified to allow the cells to mature for longer periods of time in the host prior to final challenge. There is evidence that the natural immune response to malaria parasites does vary over time and this is associated with less pathology (Brown et al, 1990; Langhorne et al, 1989; Baird, 1995). Although it is not known that a malaria parasite-specific T cell line will vary, it is likely that the numbers of cells will change and their distribution throughout the recipient host may alter to a more effective configuration. T cells were thus administered to immunodeficient BALB/c SCID mice (deficient in both T and B cells) which were then exposed to two infectious episodes, each curtailed by anti-malaria chemotherapy at 2 days post challenge with three weeks interval between infections. SCID mice were chosen so that any protection that might subsequently be observed could be ascribed to the transferred T cells. Control mice received either no T cells or T cells specific for an irrelevant antigen, ovalbumin (OVA), and which demonstrated the same phenotypic characteristics (CD4⁺, Th1). After the two infectious episodes, mice then received a final challenge which was not curtailed by chemotherapy. SCID mice which had received OVA-specific T cells all succumbed to challenge over a similar period of time as naive mice. However, two of five mice that had received the F-fraction-specific T cells survived and all recipients demonstrated significantly lower parasite densities throughout the infection (FIG. 2). Of interest, more mice that received F-specific T cells survived than mice that received T cells specific for whole parasite.

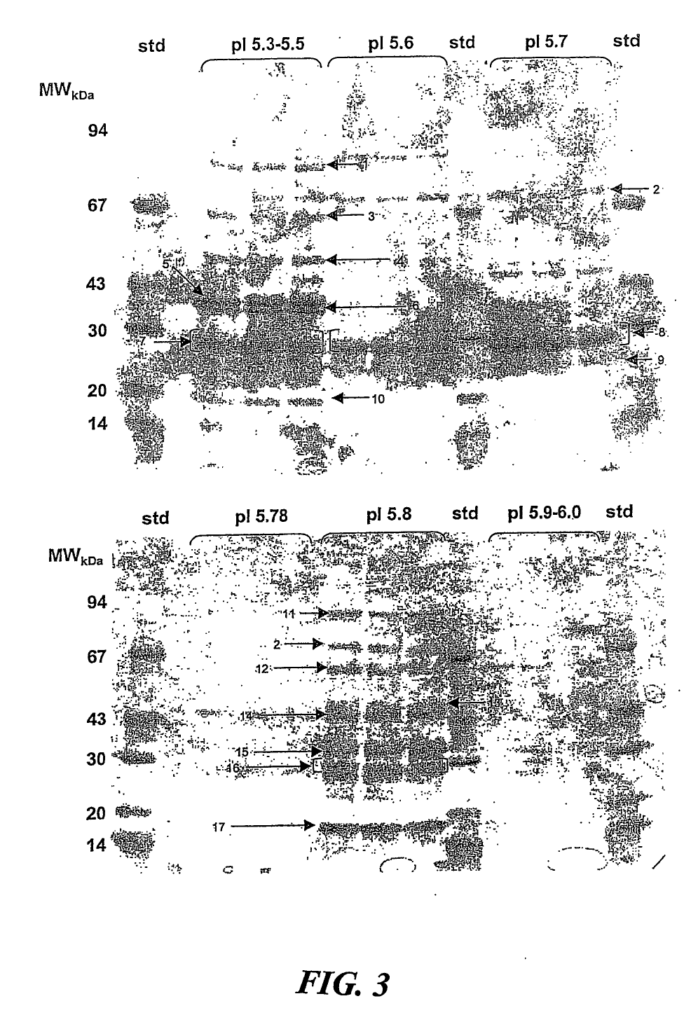

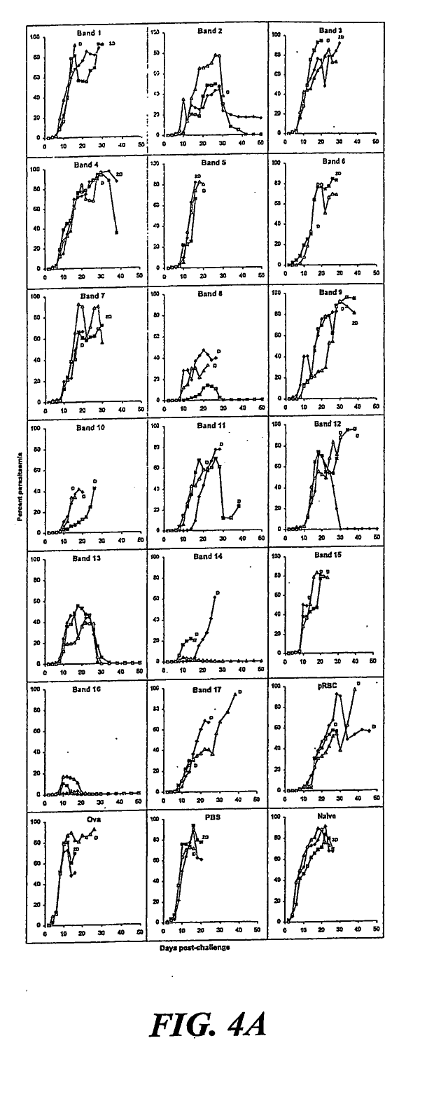

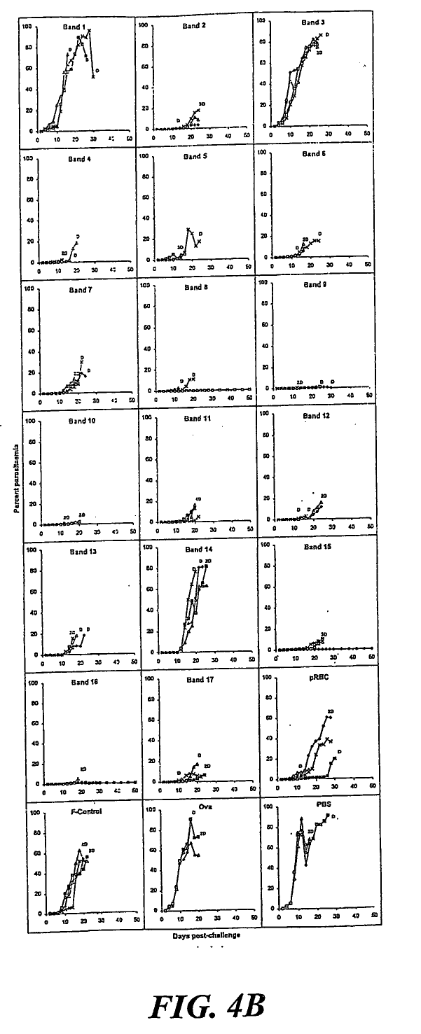

To identify the antigenic specificity(ies) of the protective T cell line, the fraction F along with adjacent fractions of similar pI were then fractionated by narrow range isoelectric focussing (pH5-7) and subsequently proteins separated by size on SDS-PAGE. Seventeen individual bands were then selected for further study (FIG. 3). Two methods were followed. In the first approach (A), new T cell lines were made by immunizing naive mice with individual bands and then expanding the cells in vitro with fraction F. These new T cell lines were then tested for specificity in vitro and for in vivo efficacy (as above). In the second approach (B), T cells specific for the fraction F were administered to SCID mice which were subsequently immunized with individual bands. Mice were then challenged with a live infection. The results of both approaches are given in FIG. 4 where it can be seen that T cells specific for band 16 were able to slow parasite growth in vivo in both approaches and protect 100% and 50% of recipients in approach A and B respectively.

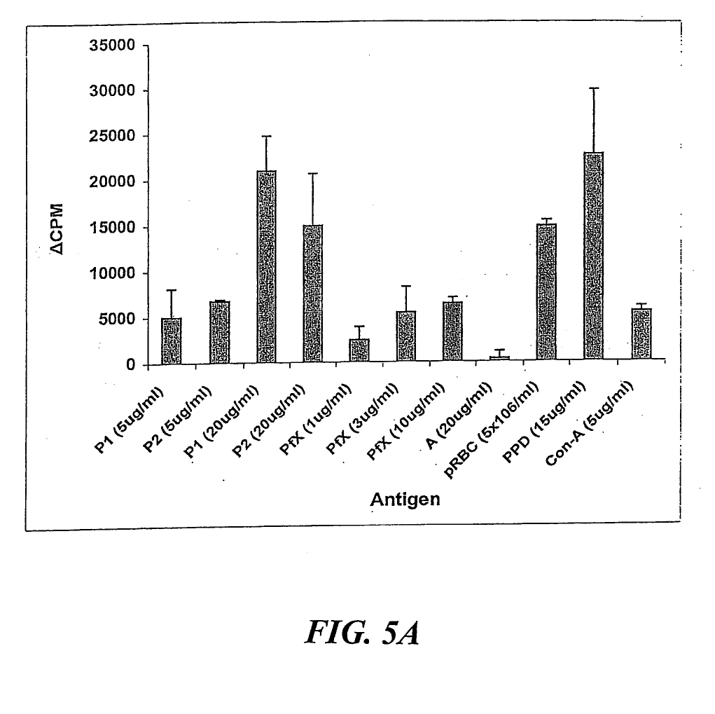

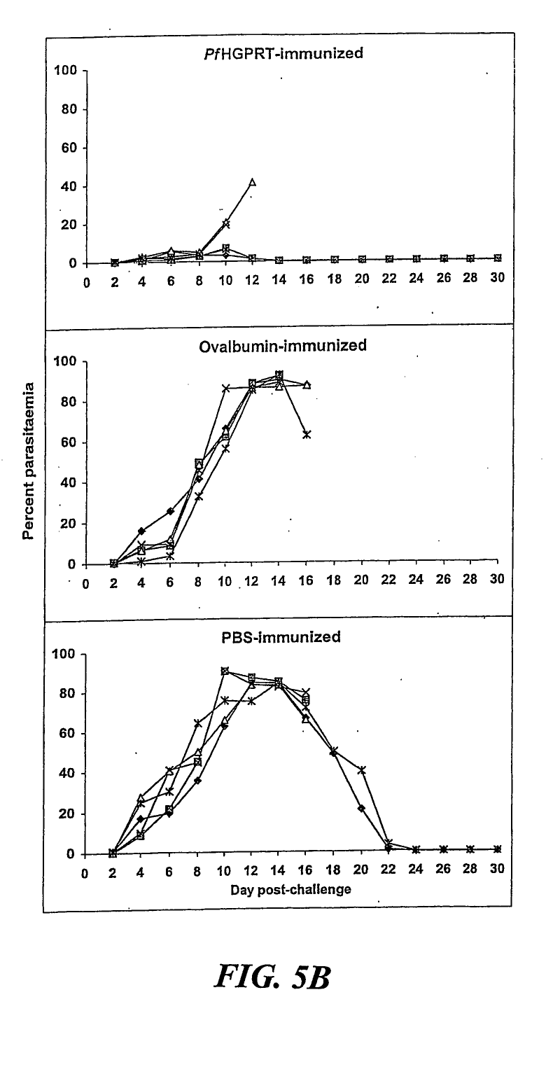

Two separate bands (“16”) of very similar molecular weight, and each associated with protection were then sequenced using the Edman degradation method of N-terminal amino acid sequencing and yielded the following sequences: MKIPNNPGAGELGYEPVMI (SEQ ID NO:49) and MKIPN (SEQ ID NO:50). From a search of the plasmodium database, we determined that these sequences are found in the purine salvage enzyme, hypoxanthine guanine xanthine phospho ribosyl transferase (HGXPRT). Recombinant falciparum HGXPRT was available (Keough et al, 1999) and tested for its ability to stimulate protective band-specific T cells. The results (FIG. 5A) demonstrated that the protective T cells did respond in vitro to the 15′ recombinant protein.

Normal BALB/c mice were then immunized with recombinant falciparum HGXPRT and challenged with a live infection. Control mice were immunized with OVA or with PBS using the same adjuvant formulation and delivery schedule. At day 8 post challenge, the mean parasitemia of immunized mice was 3.6%, compared with 43.2% in OVA-immunized mice (P<0.0001) and 47.8% PBS-immunized mice (P<0.001). Two of five HGXPRT-immunized mice were able to completely resolve their infection. There was no difference in the parasitemia of ova-immunized mice and PBS-immunized mice (P=0.545).

This study identifies the purine salvage enzyme, HGXPRT, as a target antigen of protective T cells. The efficacy of the T cells is independent of antibody as shown by the ability of T cells to adoptively transfer resistance into SCID mice. Immunization of normal mice with HGXPRT would undoubtedly induce an anti-HGXPRT antibody response but it is inconceivable that such antibodies could have an effect on parasite growth as the enzyme is located on the inner surface of the merozoite membrane (Shahabuddin et al, 1992). It would appear that the ability of HGXPRT-specific T cell transfused SCID mice and HGXPRT-immunized normal mice to control parasite growth in all recipients is due to an effect mediated by activated CD4+ T cells. Precisely how parasite-specific CD4+ T cells control parasite growth is unknown but many studies have shown that such T cells (of unknown specificity) can control parasite growth (Brake et al, 1988; Taylor-Robinson et al, 1993; Amante & Good, 1997) and many have suggested that inflammatory mediators downstream of IFN-γ and TNF-α such as nitric oxide (Taylor-Robinson et al, 1993; Rockett et al, 1991) and possibly oxygen radicals are critical (Clark & Hunt, 1983; Wozencraft et al, 1984).

This immunity in humans is characterized by induction of a parasite-specific Th1-type T cell response and up regulation of inducible nitric oxide synthase, but total lack of any measurable antibody response. T cells may be activated by parasite antigens following antigen processing by dendritic cells but the effector molecules from activated T cells and macrophages may kill parasites possibly within RBCs and in the spleen (Favila-Castillo et al, 1996). Thus, the activation of the cells is specific but the effector function may be non-specific. A side effect of the activation of parasite-specific T cells may be immunopathology (Hirunpetcharat et al, 1999), possibly explaining why not all vaccinated or T cell-transfused recipients survive, even though the parasite densities are significantly reduced in all mice.

The present inventors have previously asked whether T cells specific for the merozoite surface protein fragment, MSP 1₁₉, can protect mice (Tian et al, 1998). The results were uniformly negative despite the fact that MSP1₁₉ is capable of inducing a high degree of protection. This protection is dependent on a high antibody titer at the time of challenge. Curiously, the non-protective MSP1₁₉-specific T cells exhibited the same phenotypic characteristics (CD4+, Th1) as the protective HGXPRT-specific T cells in this study. Since T cells recognize antigen only after it is processed, it is unexplained why T cells of one specificity would protect whereas those of a different specificity would not. A possible explanation may rest with antigen localisation. HGXPRT is found in electron dense granules, similar to secretory granules of higher eukaryotes, within merozoites and is also found within the cytoplasm of the infected red cell and outside the parasitophorous vacuola (Shahabuddin et al, 1992). It is possible that antigen accumulates within the red cell cytoplasm and an antigen abundance is a critical factor in activating effector T cells.

If a conserved molecule like HGXPRT is capable of inducing T cells capable of reducing parasite density then it is also curious as to why natural immunity does not develop in children more quickly following initial exposure. Clinical immunity typically takes up to 5 years of endemic exposure to develop (Greenwood et al, 1987). It is possible that T cells of such specificity are contributing to the degree of immunity nevertheless. It is also worth noting that parasite infection is known to lead to apoptosis of human mononuclear cells (Toure-Balde et al, 1996) and indeed of parasite-specific CD4+ T cells in vivo in a rodent system (Hirunpetcharat & Good, 1998). Thus the human T cell response to HGXPRT may be suppressed by infection and not develop naturally; however, nothing is known yet of such human T cell responses. We have argued that such suppression of T cell immunity may lead to target antigens being conserved in that there is reduced immune pressure to select variant or polymorphic sequences (Good, 2001).

HGXPRT is a novel immunogen for consideration in designing a malaria vaccine and it represents a novel approach to vaccine development. HGXPRT may be useful alone as an immunogen or it may be an ideal component which could be linked to or mixed with other vaccine molecules under consideration, all of which currently are designed to stimulate a protective antibody response. By adding an additional type of immune response, as opposed to simply adding a different antigen to broaden the specificity of one type of immune response, it is likely that the chances of developing a successful vaccine will greatly improve.

### Example 2

### Mapping HGXPRT T Cell Epitopes

The data in Example 1 have indicated that Plasmodium HGXPRT may play a role in mediating protection from malaria infection. As mammals also express HGXPRT which is quite homologous to the parasite HGXPRT (FIG. 6) it is essential to identify minimal non-homologous regions of the protein that may be the target of protective T cells. These regions could potentially be used as malaria vaccine candidates.

To this end, 24 linear overlapping peptides, spanning HGXPRT were designed in an attempt to define the regions of the protein containing protective T cell epitopes (FIG. 7). Various strains of mice were immunised with either the full length recombinant P. falciparum HGXPRT protein (PfHGXPRT) or pools of the peptides derived from PfHGXPRT to determine the regions of the protein that elicit a proliferative T cell response. Following this, these regions (corresponding to peptides) were subsequently used to immunize mice to determine if the responding T cells were able to protect the mice from malaria infection. Different H-2 congenic strains of mice (inbred mice, differing only in their MHC genes) were used in these experiments to determine the role that MHC genes play in influencing the immune response to this protein.

1. Methods

Lymphocyte Proliferation Assay

Mice were immunised in the hind footpads with either pools of peptide (20 ug of each peptide) or 15 ug of recombinant PfHGXPRT protein emulsified in complete Freunds Adjuvant (CFA). Seven to nine days later, the draining popliteal and inguinal lymph nodes were removed. Cells were prepared and suspended at 2×10⁶ cells/ml in medium and added to 96 well plates. Cells were cultured with varying concentrations of antigens or mitogens for 72 hours at 37° C., 5% CO₂. The plates were pulsed with (3H)-thymidine and incorporation of radiolabel was measured 18-24 hours later by β-emission spectroscopy.

Peptide Synthesis

Peptides were synthesized by the QIMR Peptide Unit using the tea bag technology, as described (Houghten, 1985).

Peptide Immunisation and Challenge Protocol

Mice were immunised with PfHGXPRT peptides according to the following protocol. 20 ug of each peptide was emulsified in CFA and delivered sub-cutaneously (s.c) on day 0. Animals were then boosted (s.c) with 20 ug of each peptide in incomplete Freunds Adjuvant (IFA) on day 21 and then intraperitoneally on days 42 and 56. Animals were then challenged with either 1×10⁴ P. berghei or P. yoelii 17XNL pRBC on day 71. Blood samples were obtained from the tails of mice every 2 days following challenge to determine parasitaemia (the numbers of parasitized red blood cells, pRBC).

2. Results

As used herein, for convenience peptides 1-24 correspond to SEQ ID NOS:1-24.

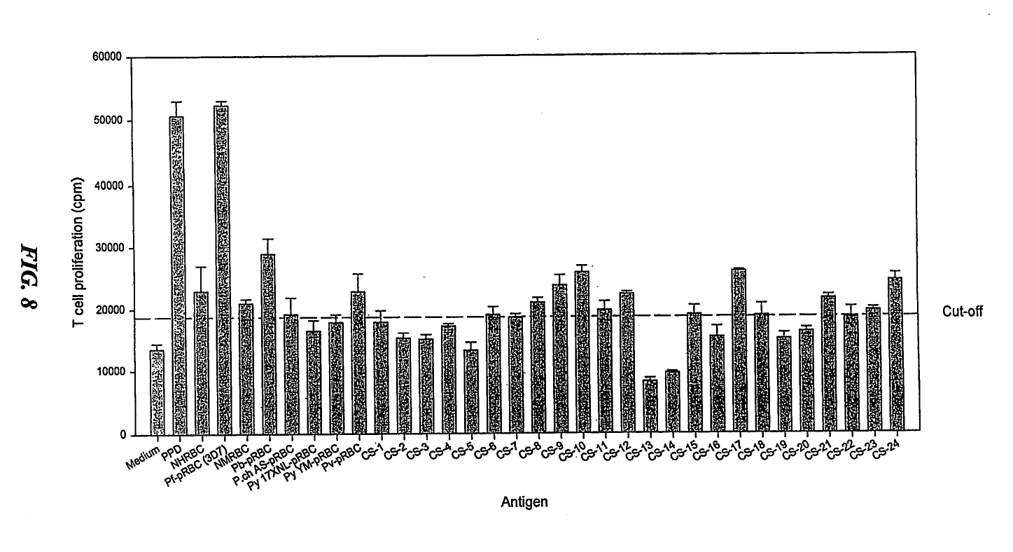

Referring to FIGS. 6-8, lymph node cells taken from BALB/c mice immunised with rPfHGXPRT respond in vitro to a number of peptides derived from rPfHGXPRT. The highest proliferative responses were observed to peptides 6, 7, 8, 9, 10, 11, 12, 15, 17, 18, 21, 22, 23 and 24. On the basis of these results, the regions of rPfHGXPRT corresponding to the above peptides may contain T cell epitopes recognised by BALB/c (H-2^{d}) mice. There were also responses to the red blood cells infected with P. falciparum and the different rodent species of Plasmodium (P. berghei, P. chabaudi AS, P. yoelii YM, P. yoelii 17XNL and P. vinckei) indicating that there is a degree of homology between HXPRT expressed by the different Plasmodium sp.

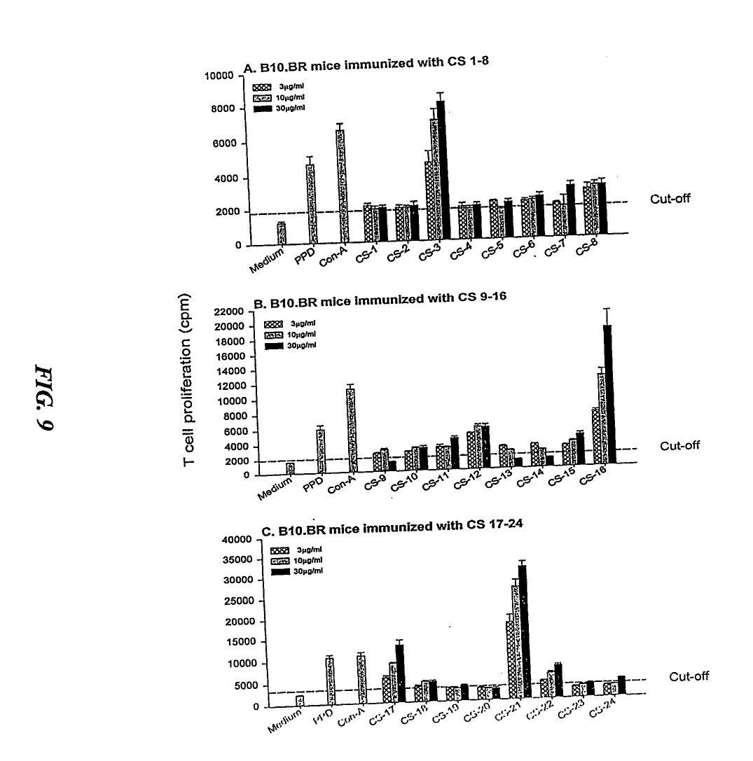

In FIG. 9A, lymph node cells from mice immunized with pool of peptides 1-8 showed strong proliferative responses to peptides 3 and 8. Low level proliferation was observed to peptides 6 and 7. Referring to FIG. 9B, lymph node cells from mice immunised with pool of peptides 9-16 showed strong proliferative responses to peptides 12 and 16. Lesser proliferative responses were observed to all other peptides in the pool. Lymph node cells from mice immunised with pool of peptides 17-24 showed strong proliferative responses to peptides 17 and 21 (FIG. 9C). Lesser proliferative responses were observed to peptide 22. In summary, the regions of PfHGXPRT corresponding to the above peptides contain T cell epitopes recognised by B10.BR (H-₂^{k}) mice.

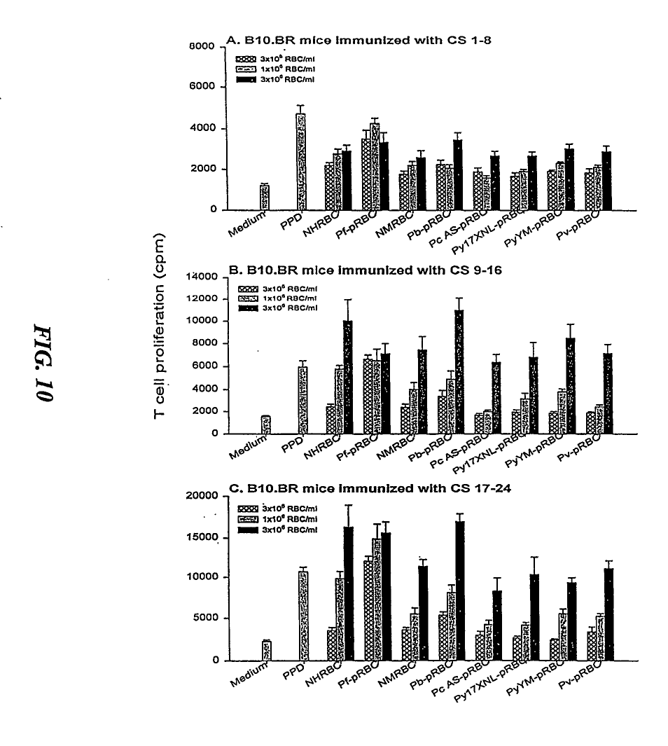

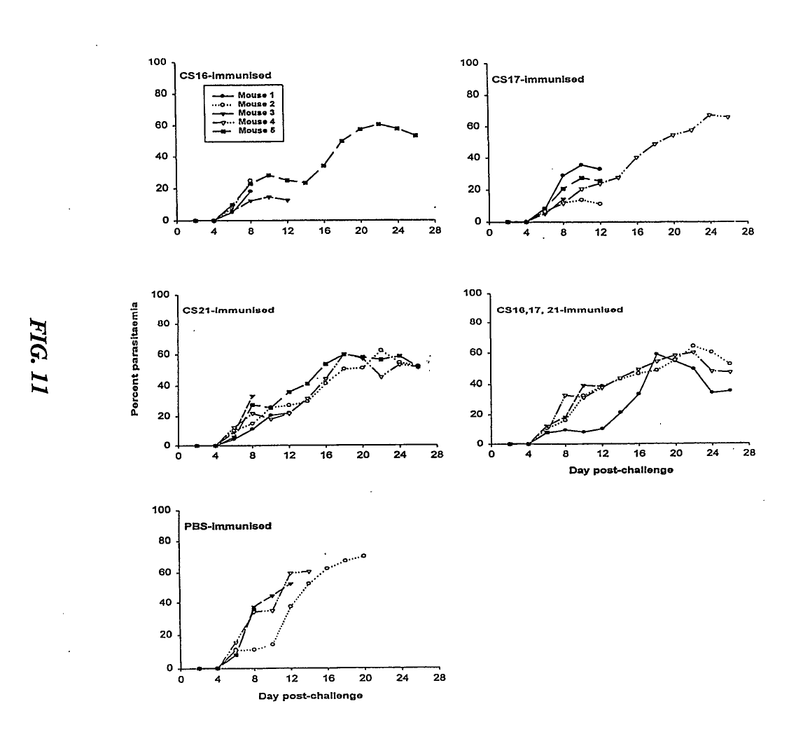

Lymph node cells from the B10.BR mice immunised with the different pools of peptides show varying levels of proliferation to not only red blood cells (RBC) infected with P. falciparum but also RBC's infected with different rodent species of Plasmodium (FIG. 10). This indicates that there is probably sequence homology of HGXPRT between the different species. However, the responses of cells from mice immunised with CS9-16 to rbc infected with P. yoelii and P. vinckei were markedly reater than to normal mouse RBC.

Naïve mice infected with P. berghei ANKA typically succumb to the disease syndrome known as cerebral malaria. Mice immunised with peptides 16 or 17 exhibited a similar course of parasitemia (or cerebral malaria) to that seen in the mice immunised with PBS. However, from FIG. 6, it appears that the mice immunised with peptides 21 or pool of peptides 16, 17 and 21 were protected from the lethal cerebral manifestations associated with P. berghei infection. These data indicate that regions of the Plasmodium enzyme HGXPRT (peptides 21 and the combination of peptides 16, 17 and 21) have been identified which contain T cell epitopes capable of inducing a protective immune response in B10.BR mice against the P. berghei ANKA parasite.

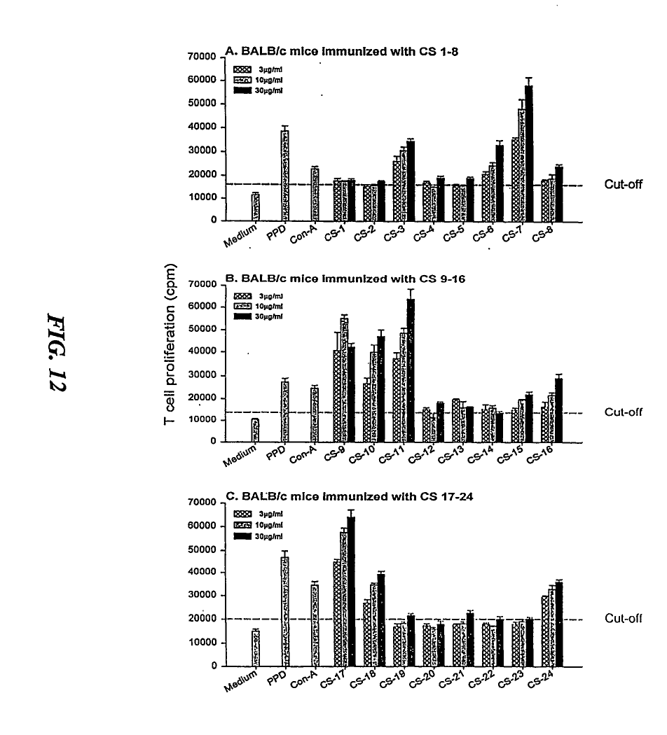

Lymph node cells from mice immunised with pooled peptides CS 1-8 showed strong proliferative responses to peptides 3, 6 and 7 (FIG. 12A). Low level proliferation was observed to peptide 8. Lymph node cells from mice immunised with pooled peptides 9-16 showed strong proliferative responses to peptides 9, 10, 11 and 16. Lesser proliferative responses were observed to peptides 13 and 15 (FIG. 12B). Referring to FIG. 12C, lymph node cells from mice immunised with pooled peptides 17-24 showed strong proliferative responses to peptides 17, 18 and 24. In summary, the regions of PfHGXPRT corresponding to the above peptides that showed proliferative responses contain T cell epitopes recognised by BALB/c (H-2^{d}) mice.

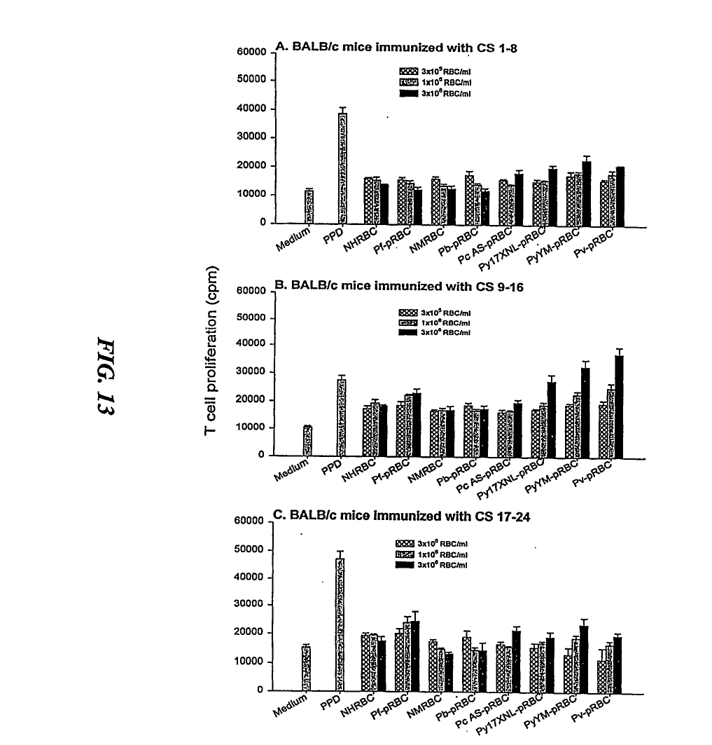

It is evident from FIG. 13 that lymph node cells from the BALB/c mice immunised with the different pools of peptides show varying levels of proliferation to not only red blood cells (rbc) infected with P. falciparum but also rbc infected with different rodent species of Plasmodium. This indicates that there is probably sequence homology of HGXPRT between the different species.

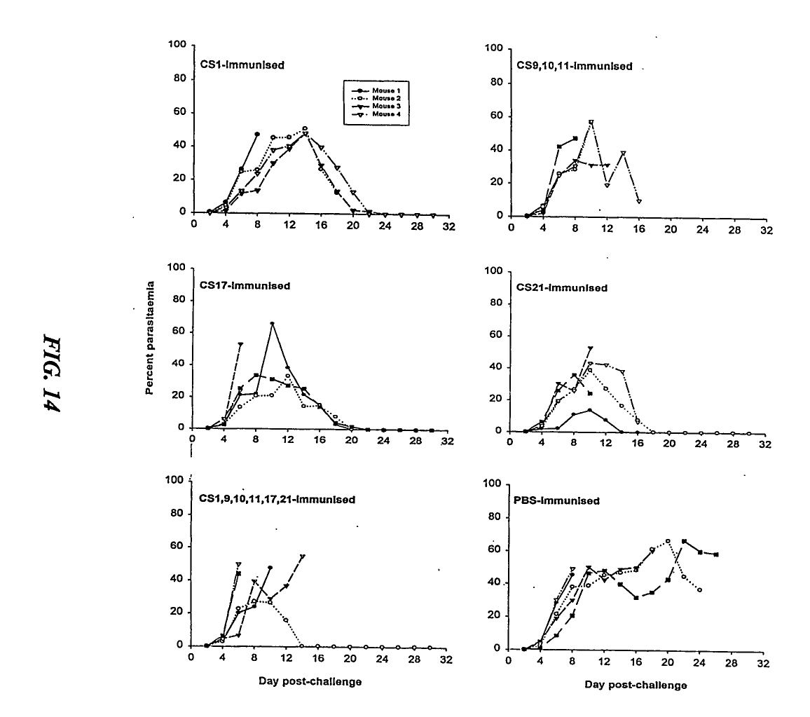

In FIG. 14, peptides 1, 17 or 21 acted in a curative manner, offering a degree of protection to mice challenged with P. yoelii 17XNL, although one mouse from each group succumbed to challenge. Pooled peptides 9, 10 and 11 and pooled peptides 1, 9, 10, 11, 17 and 21 did not protect the mice from lethal challenge with P. yoelii 17XNL. These data indicate that regions of the Plasmodium enzyme HGXPRT (peptides 1, 17 and 21) have been identified which contain T cell epitopes capable of inducing a protective immune response in BALB/c mice against the P. yoelii 17NXL parasite.

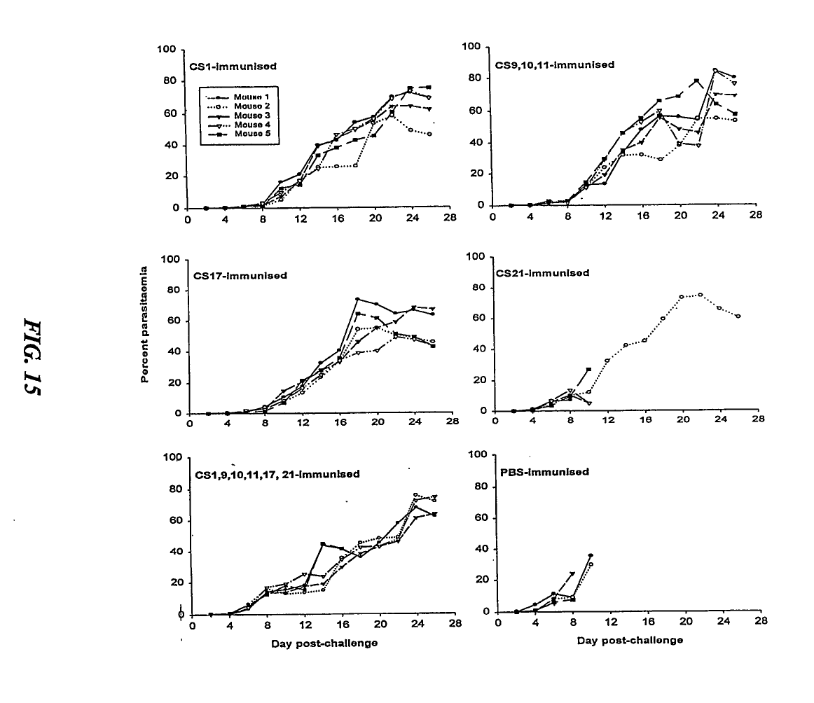

In FIG. 15, Mice that were immunised with either peptide 1, pooled peptides 9, 10 and 11, peptide 17 or pooled peptides 1, 9, 10, 11, 17 and 21 were protected from the lethal cerebral manifestations associated with P. berghei ANKA infection. These peptides appear to induce an anti-disease immune response. Mice immunised with peptide 21 and control mice immunised with PBS were not protected and succumbed to challenge. These data indicate that regions of the Plasmodium enzyme HGXPRT (peptides 1, 17, the combination of peptides 9, 10, 11 and the combination of peptides 1, 9, 10, 11, 17 and 21) have been identified which contain T cell epitopes capable of inducing a protective immune response in BALB/c mice against the P. berghei ANKA parasite.

In further experiments aimed at investigating MHC restriction, BALB/b mice (H-2^{b}) responded to peptides 1, 7, 8, 10, 15, 17, 18, 19, 20, 21, 22, 23 and 24; BIO mice (H-2^{b}) responded to peptides 4, 5, 6, 7, 8, 10, 15, 16, 17, 18, 19, 20, 21, 22, 23 and 24.; B10.D2 mice (H-2^{d}) responded to peptides 1, 2, 3, 4, 7, 8, 9, 10, 11, 15, 16, 17, 18, 22 and 24; BALB/c mice (H-2^{d}) (see FIG. 12) respond to peptides 3, 6, 7, 8, 9, 10, 11, 13, 15, 16, 17, 18 and 24; BALB/k mice (H-2^{k}) responded to peptides 3, 4, 5, 6, 7, 8, 12, 15, 16, 17, 21 and 22; and B10.BR mice (H-2^{k}) respond to peptides 3, 6, 8, 10, 11, 12, 15, 16, 17, 21 and 22.

We conclude that the MHC genes may influence the immune response to some of the peptides derived from HGXPRT. It is also possible that regions outside the MHC may play a role. Further work remains to be done to clarify this.

Furthermore, lymph node cells from the BALB/k mice, B10 mice, B10.D2 mice and BALB/b mice immunised with the different pools of peptides showed varying levels of proliferation to not only red blood cells (rbc) infected with P. falciparum but also rbc infected with different rodent species of Plasmodium. This indicates that there is probably sequence homology of HGXPRT between the different species.

### SUMMARY

Studies performed with overlapping peptides derived from P. falciparum HGXPRT and various strains of inbred mice have identified the regions of HGXPRT that are the target of T cells. Additionally, it has been demonstrated that small regions of this protein (corresponding to particular peptides) can induce an immune response capable of controlling the growth of the rodent parasite strains tested (P. yoelii 17XNL and P. berghei ANKA) and the disease symptoms associated with rodent malaria infection. The regions corresponding to peptides CS17 and CS21 are of particular interest as they are able to induce a protective immune response against different rodent parasites in multiple mouse strains. These peptides should be considered as malaria vaccine candidates.

Throughout this specification, the aim has been to describe the preferred embodiments of the invention without limiting the invention to any one embodiment or specific collection of features. Various changes and modifications may be made to the embodiments described and illustrated herein without departing from the broad spirit and scope of the invention.

All computer programs, algorithms, patent and scientific literature referred to in this specification are incorporated herein by reference in their entirety.

### REFERENCES

Freeman R R, Parish C R. Plasmodium yoelii: antibody and the maintenance of immunity in BALB/c mice. Exp Parasitol. 1981 August;52(1):18-24.
Grun J L, Weidanz W P. Antibody-independent immunity to reinfection malaria in B-cell-deficient mice. Infect Immun. 1983 September;41(3): 1197-204.
Brake D A, Long C A, Weidanz W P. Adoptive protection against Plasmodium chabaudi adami malaria in athymic nude mice by a cloned T cell line. J. Immunol. 1988 Mar. 15;140(6):1989-93.
Seixas E M, Langhorne J. gammadelta T cells contribute to control of chronic parasitemia in Plasmodium chabaudi infections in mice. J. Immunol. 1999 Mar. 1;162(5):2837-41.
Good, M. F. Towards a blood stage vaccine for malaria: Are we following all the leads? Nature Rev. Immunol. 1: 117-125, 2001.
Brown A E, Webster H K, Teja-Isavadharm P, Keeratithakul D. Macrophage activation in falciparum malaria as measured by neopterin and interferon-gamma. Clin Exp Immunol. 1990 October;82(1):97-101.
Langhorne J, Gillard S, Simon B, Slade S, Eichmann K. Frequencies of CD4+ T cells reactive with Plasmodium chabaudi chabaudi: distinct response kinetics for cells with Th1 and Th2 characteristics during infection. Int Immunol. 1989;1(4):416-24.
Baird J K. Host age as a determinant of naturally acquired immunity to Plasmodium falciparum. 1995. Parasitol Today 11:105-111
Keough D T, N g A L, Winzor D J, Emmerson B T, de Jersey J. Purification and characterization of Plasmodium falciparum hypoxanthine-guanine-xanthine phosphoribosyltransferase and comparison with the human enzyme. Mol Biochem Parasitol. 1999 Jan. 5;98(1):29-41.
Shahabuddin M, Gunther K, Lingelbach K, Aikawa M, Schreiber M, Ridley R G, Scaife J G. Localisation of hypoxanthine phosphoribosyl transferase in the malaria parasite Plasmodium falciparum. Exp Parasitol. 1992 February;74(1):11-9.
Brake D A, Long C A, Weidanz W P. Adoptive protection against Plasmodium chabaudi adami malaria in athymic nude mice by a cloned T cell line. J. Immunol. 1988 Mar. 15;140(6):1989-93.
Taylor-Robinson A W, Phillips R S, Severn A, Moncada S, Liew F Y. The role of TH1 and TH2 cells in a rodent malaria infection. Science. 1993 Jun. 25;260(5116): 1931-4.
Amante F H, Good M F. Prolonged Th1-like response generated by a Plasmodium yoelii-specific T cell clone allows complete clearance of infection in reconstituted mice. Parasite Immunol. 1997 March;19(3):111-26.
Rockett K A, Awburn M M, Cowden W B, Clark I A. Killing of Plasmodium falciparum in vitro by nitric oxide derivatives. Infect Immun. 1991 September;59(9):3280-3.
Clark I A, Hunt N H. Evidence for reactive oxygen intermediates causing hemolysis and parasite death in malaria. Infect Immun. 1983 January;39(1):1-6. Favila-Castillo L, Monroy-Ostria A, Kobayashi E, Hirunpetcharat C, Kamada N,
Good M F. Protection of rats against malaria by a transplanted immune spleen. Parasite Immunol. 1996 July; 18(7):325-31.
Hirunpetcharat C, Finkelman F, Clark I A, Good M F. Malaria parasite-specific Th1-like T cells simultaneously reduce parasitemia and promote disease. Parasite Immunol. 1999 June;21(6):319-29.
Tian J H, Good M F, Hirunpetcharat C, Kumar S, Ling I T, Jackson D, Cooper J, Lukszo J, Coligan J, Ahlers J, Saul A, Berzofsky J A, Holder A A, Miller L H, Kaslow D C. Definition of T cell epitopes within the 19 kDa carboxylterminal fragment of Plasmodium yoelii merozoite surface protein 1 (MSP1(19)) and their role in immunity to malaria. Parasite Immunol. 1998 June;20(6):263-78.
Greenwood B M, Bradley A K, Greenwood A M, Byass P, Jammeh K, Marsh K, Tulloch S, Oldfield F S, Hayes R. Mortality and morbidity from malaria among children in a rural area of The Gambia, West Africa. Trans R Soc Trop Med Hyg. 1987;81(3):478-86.
Toure-Balde A, Sarthou J L, Aribot G, Michel P, Trape J F, Rogier C, Roussilhon C. Plasmodium falciparum induces apoptosis in human mononuclear cells. Infect Immun. 1996 March;64(3):744-50.
Hirunpetcharat C, Good M F. Deletion of Plasmodium berghei-specific CD4+ T cells adoptively transferred into recipient mice after challenge with homologous parasite. Proc Natl Acad Sci USA. 1998 Feb. 17;95(4):1715-20.
Sander B, Hoiden I, Andersson U, Moller E and Abrams JS (1993): Similar frequencies and kinetics of cytokine producing cells in murine peripheral blood and spleen. Cytokine detection by immunoassay and intracellular immunostaining. J Immunol Methods 166 (2): 201-14.
Laemmli U K (1970): Cleavage of structural proteins during the assembly of the head of bacteriophage T4. Nature 227(259):680-5.
Dainese P, Hoyer-Hansen G and Bassi R (1990): The resolution of chlorophyll a/b binding proteins by a preparative method based on flat bed isoelectric focusing. Photochem Photobiol 51(6):693-703.
Edman P and Begg G (1967): A protein sequenator. Eur J Biochem 1(1):80-91.
Narum D L, Kumar S, Rogers W O, Fuhrmann SR, Liang H, Oakley L, Taye A, Sim B K and Hoffman S L (2001): Codon optimization of gene fragments encoding Plasmodium falciparum merozoite proteins enhances DNA vaccine protein expression and immungenicity in mice., Infect. Immun. 69 (12): 7250-3.
Kumar S, Epstein J E, Richie T L, Nkrumah F K, Soisson L, Carucci D and Hoffman SL (2002): A multilateral effort to develop DNA vaccines against falciparum malaris. Trends Parasitol. 18(3): 129-35.
Doolan D L & Hoffman S L (2001): DNA-based vaccines against malaria: status and promise of the Multi-Stage Malaria DNA Vaccine Operation. Int. J. Parasitol. 31(8):753-62.
Boyle et al (1998): Nature 393: 408-411.
Houghten, R. A. (1985) General method for the rapid solid-phase synthesis of large numbers of peptides: specificity of antigen-antibody interaction at the level of individual amino acids, Proc Natl Acad Sci USA, 82(15), pp. 5131-5135.
Dressel A, Chin J L, Sette A, Gausling R, Hollsberg P, Hafler D A. (1997) Autoantigen recognition by human CD8 T cell clones: enhanced agonist response induced by altered peptide ligands. J. Immunol. 159(10) pp. 4943-4951.
Michielin et al. (2000). Modeling of the TCR-MHC-peptide complex. J. Mol. Biol. 300 (5) pp 1205-1235.
Hanke T, Schneider J, Gilbert S C, Hill A V, McMichael A (1999) DNA multi-CTL epitope vaccines for HIV and Plasmodium falciparum: immunogenicity in mice. Vaccine 16(4) pp. 426-35.

1. An isolated protein comprising at least one immunogenic fragment of a hypoxanthine guanine xanthine phosphoribosyl transferase (HGXPRT) protein, wherein the isolated protein is not a full-length HGXPRT.

2. The isolated protein of claim 1, comprising an amino acid sequence selected from the group consisting of: SEQ ID NO: 1; SEQ ID NO: 2; SEQ ID NO: 3; SEQ ID NO: 4; SEQ ID NO: 5; SEQ ID NO: 6; SEQ ID NO:7; SEQ ID NO: 8; SEQ ID NO:9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; and SEQ ID NO: 24.

3. The isolated protein of claim 1, comprising an amino acid sequence selected from the group consisting of SEQ ID NO: 6; SEQ ID NO: 7, SEQ ID NO: 8, SEQ ID NO: 9, SEQ ID NO: 10, SEQ ID NO: 11, SEQ ID NO: 12, SEQ ID NO: 15, SEQ ID NO: 17, SEQ ID NO: 18, SEQ ID NO: 21, SEQ ID NO: 22, SEQ ID NO: 23 and SEQ ID NO: 24.

4. The isolated protein of claim 1, comprising an amino acid sequence set forth in SEQ ID NO: 17 or SEQ ID NO: 21.

5. An isolated protein comprising a plurality of immunogenic fragments of a hypoxanthine guanine xanthine phosphoribosyl transferase (HGXPRT) protein, wherein the isolated protein is not a full-length HGXPRT protein.

6. An immunotherapeutic composition comprising an isolated hypoxanthine guanine xanthine phosphoribosyl transferase (HGXPRT) protein, or one or more isolated proteins each comprising at least one immunogenic fragment thereof, and an immunologically-acceptable carrier, diluent or excipient.

7. The immunotherapeutic composition of claim 6, wherein the isolated protein comprises an amino acid sequence selected from the group consisting of: SEQ ID NO: 1; SEQ ID NO: 2 SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO:11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; and SEQ ID NO: 24.

8. The immunotherapeutic composition of claim 6, wherein the isolated protein has an amino acid sequence selected from the group consisting of SEQ ID NO: 6; SEQ ID NO: 7; SEQ ID NO: 8; SEQ ID NO: 9; SEQ ID NO: 10; SEQ ID NO:11; SEQ ID NO: 12; SEQ ID NO: 15; SEQ ID NO: 17; SEQ ID NO: 18; SEQ ID NO: 21; SEQ ID NO: 22; SEQ ID NO: 23 and SEQ ID NO: 24.

9. The immunotherapeutic composition of claim 6, wherein the isolated protein has an amino acid sequence set forth in SEQ ID NO: 17 or SEQ ID NO: 21.

10. The immunotherapeutic composition of claim 6, further comprising one or more B cell epitopes.

11. The immunotherapeutic composition of claim 10, wherein the one or more B cell epitopes are derived from the carboxy terminus of MSP1 or Apical membrane Antigen 1.

12. An isolated nucleic acid encoding the isolated protein of claim 1.

13. The isolated nucleic acid of claim 12, comprising a nucleotide sequence selected from the group consisting of: SEQ ID NO: 25; SEQ ID NO: 26; SEQ ID NO: 27; SEQ ID NO: 28; SEQ ID NO: 29; SEQ ID NO: 30; SEQ ID NO: 31; SEQ ID NO: 32; SEQ ID NO: 33; SEQ ID NO: 34; SEQ ID NO: 35; SEQ ID NO: 36; SEQ ID NO: 37; SEQ ID NO: 38; SEQ NO: 39; SEQ ID NO: 40; SEQ ID NO: 41; SEQ ID NO: 42; SEQ ID NO: 43; SEQ ID NO: 44; SEQ NO: 45; SEQ ID NO: 46; SEQ ID NO: 47; and SEQ ID NO: 48.

14. A polyepitope construct comprising an isolated nucleic acid encoding a plurality of the isolated proteins of claim 1.

15. An immunotherapeutic composition comprising an isolated nucleic acid encoding a hypoxanthine guanine xanthine phosphoribosyl transferase (HGXPRT) protein, or at least one immunogenic fragment thereof, and an immunologically-acceptable carrier, diluent or excipient.

16. The immunotherapeutic composition of claim 15, wherein the isolated nucleic acid encodes an amino acid sequence selected from the group consisting of: SEQ ID NO: 1; SEQ ID NO: 2 SEQ ID NO: 3; SEQ ID NO: 4; SEQ ID NO: 5; SEQ ID NO: 6; SEQ ID NO: 7; SEQ ID NO: 8; SEQ ID NO:9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; and SEQ ID NO: 24.

17. The immunotherapeutic composition of claim 15, comprising a nucleotide sequence selected from the group consisting of SEQ ID NO: 25; SEQ ID NO: 26; SEQ ID NO: 27; SEQ ID NO: 28; SEQ ID NO: 29; SEQ ID NO: 30; SEQ ID NO: 31; SEQ ID NO: 32; SEQ ID NO: 33; SEQ ID NO: 34; SEQ ID NO: 35; SEQ ID NO: 36; SEQ ID NO: 37; SEQ ID NO: 38; SEQ NO: 39; SEQ ID NO: 40; SEQ ID NO: 41; SEQ ID NO: 42; SEQ ID NO: 43; SEQ ID NO: 44; SEQ NO: 45; SEQ ID NO: 46; SEQ ID NO: 47; and SEQ ID NO: 48.

18. The immunotherapeutic composition of claim 6, which elicits an immune response against a disease-causing protozoan.

19. The immunotherapeutic composition of claim 18, wherein the protozoan is a Plasmodium species.

20. An isolated T-lymphocyte that recognizes an immunogenic fragment of a HGXPRT protein.

21. The isolated T-lymphocyte of claim 20, which is a CD4+ T lymphocyte.

22. A non-human animal immunized with an isolated HGXPRT protein or at least one immunogenic fragment thereof.

23. A method of immunization against a protozoal disease, said method including the step of administering a HGXPRT protein, or at least one immunogenic fragment thereof, to an animal.

24. A method of immunization against a protozoal disease, said method including the step of administering an isolated nucleic acid encoding a HGXPRT protein or at least one immunogenic fragment thereof, to an animal.

25. The method of claim 23, wherein the of immunogenic fragment comprises an amino acid sequence selected from the group consisting of: SEQ ID NO: 1; SEQ ID NO: 2 SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; and SEQ ID NO: 24.

26. The method of claim 25, wherein the immunogenic fragment comprises an amino acid sequence selected from the group consisting of SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 15; SEQ ID NO: 17; SEQ ID NO: 18; SEQ ID NO: 21; SEQ ID NO: 22; SEQ ID NO: 23 and SEQ ID NO: 24.

27. The method of claim 26, wherein the or each immunogenic fragment comprises an amino acid sequence set forth in SEQ ID NO: 17 or SEQ ID NO: 21.

28. The method of claim 23, which elicits an immune response against a disease-causing protozoan.

29. The method of claim 28, wherein the protozoan is a Plasmodium species.

30. The method of claim 23, wherein the animal is a mammal.

31. The method of claim 30, wherein the mammal is a human.

32. An antibody which binds an immunogenic fragment of a hypoxanthine guanine xanthine phosphoribosyl transferase (HGXPRT) protein.

33. The antibody of claim 32, which binds an amino acid sequence selected from the group consisting of: SEQ ID NO: 1; SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; and SEQ ID NO: 24.

34. The immunotherapeutic composition of claim 15, which elicits an immune response against a disease-causing protozoan.

35. The immunotherapeutic composition of claim 34, wherein the protozoan is a Plasmodium species.

36. The method of claim 24, wherein the immunogenic fragment comprises an amino acid sequence selected from the group consisting of: SEQ ID NO: 1; SEQ ID NO: 2 SEQ ID NO: 3; SEQ ID NO: 4; SEQ ID NO: 5; SEQ ID NO: 6; SEQ ID NO: 7; SEQ ID NO: 8; SEQ ID NO:9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; and SEQ ID NO: 24.

37. The method of claim 36, wherein the immunogenic fragment comprises an amino acid sequence selected from the group consisting of SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 15; SEQ-ID NO: 17; SEQ ID NO: 18; SEQ ID NO: 21; SEQ ID NO: 22; SEQ ID NO: 23 and SEQ ID NO: 24.

38. The method of claim 24, which elicits an immune response against a disease-causing protozoan.

39. The method of claim 38, wherein the protozoan is a Plasmodium species.

40. The method of claim 24, wherein the animal is a mammal.

41. The method of claim 40, wherein the mammal is a human.
